(19) United States
(12) Patent Application Publication
(10) Pub. No.: US 2023/0117850 A1
(43) Pub. Date: Apr. 20, 2023

(54) FUNCTIONAL MEMBER AND CHEMICAL SUBSTANCE SENSOR INCLUDING THE SAME

(71) Applicant: Panasonic Intellectual Property Managenent Co., Ltd., Osaka (JP)

(72) Inventors: TEPPEI HOSOKAWA, Hyogo (JP); TOMOKO KAWASHIMA, Osaka (JP); YUKO TANIIKE, Osaka (JP)

(21) Appl. No.: 18/064,321

(22) Filed: Dec. 12, 2022

Related U.S. Application Data
(63) Continuation of application No. PCT/JP2021/024433, filed on Jun. 29, 2021.

(30) Foreign Application Priority Data
Jul. 2, 2020 (JP) 2020-115125
Jul. 2, 2020 (JP) 2020-115127
Jun. 8, 2021 (JP) 2021-096147

(51) Int. Cl. G01N 21/64; C07C 63/28; C01C 1/00; C08J 5/18

(52) U.S. Cl. CPC G01N 21/6486; G01N 21/645; C07C 63/28; C01C 1/00; C08J 5/18; G01N 2021/6482; C08J 2301/02

(57) ABSTRACT

A functional member includes a porous member with a cavity and a trapping agent that traps a chemical substance. The trapping agent is held in the cavity of the porous member.

### BACKGROUND

### 1. Technical Field

The present disclosure relates to a functional member and a chemical substance sensor including the functional member.

### 2. Description of the Related Art

Organic salts that trap chemical substances are known. An example of organic salts is an organic salt formed by ionic bonding between an organic acid molecule with a carboxy group or a sulfonate group and an amine molecule with an amino group. International Publication No. WO 2019/244464 disclosed by the present applicant discloses an organic salt containing terephthalic acid and a primary alkylamine. International Publication No. WO 2019/244464 discloses that the organic salt chemically adsorbs a hydroxyl radical and can detect the hydroxyl radical by changing its fluorescence properties due to the adsorption. Furthermore, International Publication Nos. WO 2018/169022 and WO 2018/169023 disclosed by the present applicant and HOSOKAWA Teppei et al., “Application of Organic Composite Composed of Carboxylic Acid Amine Salt to Ammonia Sensor”, proceedings of 68th Annual Meeting of the Society of Polymer Science (2019), Session ID: 3J16 (hereinafter abbreviated to Non-patent Literature 1) disclose an organic salt containing a cyanoacrylic acid derivative and triphenylmethylamine. These pieces of literature disclose that the organic salt physically adsorbs ammonia and can detect the ammonia by changing its fluorescence properties due to the adsorption.

### SUMMARY

In one general aspect, the techniques disclosed here feature a functional member a porous member with a cavity, and a trapping agent that is held in the cavity and that traps a chemical substance.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a schematic view of an example of a functional member according to the present disclosure;

FIG. 2 is a graph of an example of an X-ray diffraction pattern of natural cellulose;

FIG. 3 illustrates an example of a primary alkylamine that may be contained in an organic salt A, which is an example of a trapping agent;

FIG. 4 illustrates an example of a cyanoacrylic acid derivative that may be contained in an organic salt B, which is an example of a trapping agent;

FIG. 5 illustrates an example of a trisubstituted methylamine that may be contained in the organic salt B, which is an example of a trapping agent;

FIG. 6 is a schematic cross-sectional view of an example of a chemical substance sensor according to the present disclosure;

FIG. 7 is a schematic exploded perspective view of another example of a chemical substance sensor according to the present disclosure;

FIG. 8 is a schematic exploded perspective view of still another example of a chemical substance sensor according to the present disclosure;

FIG. 9 is a schematic exploded perspective view of still another example of a chemical substance sensor according to the present disclosure;

FIG. 10A is a schematic exploded view of another example of a chemical substance sensor according to the present disclosure;

FIG. 10B is a cross-sectional view of a fixing member and a magnet in a lid portion taken along the line XB-XB of FIG. 10A;

FIG. 11 is a schematic exploded view of another example of a chemical substance sensor according to the present disclosure;

FIG. 12 is a schematic view of an example of the use of a chemical substance sensor according to the present disclosure;

FIG. 13 is a graph of X-ray diffraction patterns of an organic salt and a functional sheet prepared in Example 1;

FIG. 14A is an enlarged observation image of the functional sheet prepared in Example 1 acquired with a scanning electron microscope;

FIG. 14B is an enlarged image of a region R2 in the enlarged observation image of FIG. 14A;

FIG. 14C is an enlarged image of a region R3 in the enlarged observation image of FIG. 14B;

FIG. 15A is an enlarged observation image of a portion different from FIG. 14A in the functional sheet prepared in Example 1 acquired with a scanning electron microscope;

FIG. 15B is an enlarged image of a region R4 in the enlarged observation image of FIG. 15A;

FIG. 15C is an enlarged image of a region R5 in the enlarged observation image of FIG. 15B;

FIG. 16A is a schematic view of a chamber used to expose a functional sheet to an atmosphere containing hydroxyl radicals in Examples 1 to 3 and Comparative Example 1;

FIG. 16B is a photograph of the chamber used in Examples 1 to 3 and Comparative Example 1 taken from a point X located obliquely above the chamber;

FIG. 17 is a fluorescence image A and a fluorescence image B of the functional sheet prepared in Example 1;

FIG. 18 is a fluorescence image A′ and a fluorescence image B′ of the functional sheet prepared in Example 1;

FIG. 19 is a graph of a fluorescence spectrum emitted from a solution of an organic salt extracted from a functional sheet after exposure to an atmosphere containing hydroxyl radicals in Example 1;

FIG. 20 is a graph of X-ray diffraction patterns of an organic salt and a functional sheet prepared in Example 2;

FIG. 21 is a fluorescence image A and a fluorescence image B of the functional sheet prepared in Example 2;

FIG. 22 is a fluorescence image A′ and a fluorescence image B′ of the functional sheet prepared in Example 2;

FIG. 23 is a graph of a fluorescence spectrum emitted from a solution of an organic salt extracted from a functional sheet after exposure to an atmosphere containing hydroxyl radicals in Example 2;

FIG. 24 is a graph of X-ray diffraction patterns of an organic salt and a functional sheet prepared in Example 3;

FIG. 25 is a fluorescence image A and a fluorescence image B of the functional sheet prepared in Example 3;

FIG. 26 is a fluorescence image A′ and a fluorescence image B′ of the functional sheet prepared in Example 3;

FIG. 27 is a graph of a fluorescence spectrum emitted from a solution of an organic salt extracted from a functional sheet after exposure to an atmosphere containing hydroxyl radicals in Example 3;

FIG. 28 is a fluorescence image A and a fluorescence image B of a pellet prepared in Comparative Example 1;

FIG. 29 is a graph of a fluorescence spectrum emitted from a solution of an organic salt prepared by dissolving a pellet after exposure to an atmosphere containing hydroxyl radicals in Comparative Example 1;

FIG. 30 is a photograph of a chamber actually used to expose a functional sheet to an atmosphere containing hydroxyl radicals and the exposure state in Example 4;

FIG. 31 is a fluorescence image A and a fluorescence image B of each functional sheet prepared in Example 4;

FIG. 32 is a graph of a fluorescence spectrum emitted from a solution of an organic salt extracted from each functional sheet after exposure to an atmosphere containing hydroxyl radicals in Example 4;

FIG. 33 is a photograph of a chamber actually used to expose a functional sheet to an atmosphere containing hydroxyl radicals and the exposure state in Example 5;

FIG. 34 is a fluorescence image A and a fluorescence image B of the functional sheet prepared in Example 5;

FIG. 35 is a graph of a fluorescence spectrum emitted from a solution of an organic salt extracted from a functional sheet after exposure to an atmosphere containing hydroxyl radicals in Example 5;

FIG. 36 is a graph of the relationship between the holding time of a functional sheet in a body surface gas exposure test in Example 6 and the difference D between the brightness values of Blue fluorescence emitted from the functional sheet before and after the holding;

FIG. 37 is a graph of the relationship between the holding time of the functional sheet in the body surface gas exposure test in Example 6 and the difference D₁-D₂ between the difference D₁ of a first sheet held in contact with a subject and the difference D₂ of a second sheet held beside the subject, each of the differences D₁ and D₂ being the difference D between the brightness values of Blue fluorescence emitted from the functional sheet before and after the holding;

FIG. 38 is a graph of the relationship between the holding time of a functional sheet in a body surface gas exposure test in Example 8 and the difference D between the brightness values of Blue fluorescence emitted from the functional sheet before and after the holding;

FIG. 39 is a graph of the relationship between the holding time of the functional sheet in the body surface gas exposure test in Example 8 and the difference D₁-D₂ between the difference D₁ of a first sheet in a first sensor held in contact with a subject and the difference D₂ of a second sheet in a second sensor held beside the subject, each of the differences D₁ and D₂ being the difference D between the brightness values of Blue fluorescence emitted from the functional sheet before and after the holding;

FIG. 40 is a graph of X-ray diffraction patterns of an organic salt and a functional sheet prepared in Example 9;

FIG. 41 is a schematic explanatory view of a chamber used to expose a functional sheet to an atmosphere containing ammonia in Example 9, the exposure state, and a method for photographing fluorescence emitted from the functional sheet;

FIG. 42 is a graph of the relationship between the elapsed time in an exposure test to the atmosphere containing ammonia in Example 9 and the rate of change in the brightness of Green fluorescence emitted from a functional sheet;

FIG. 43 is a graph of the relationship between the transmittance of light with a wavelength of 450 nm and the hydroxyl radical detection efficiency in functional sheets according to Examples 11 to 13 and a pellet according to Comparative Example 12; and

FIG. 44 is a view of the state of fluorescence emission before and after ultraviolet irradiation on an exposed surface and a back surface of a functional sheet according to Example 13.

### DETAILED DESCRIPTIONS

International Publication Nos. WO 2019/244464, WO 2018/169022, and WO 2018/169023 and Non-patent Literature 1 describe the detection of a chemical substance using pellet-like organic salt crystals. However, it is difficult to easily detect a chemical substance with high sensitivity by this method.

One non-limiting and exemplary embodiment provides a technique that can easily detect a chemical substance, such as the organic salt described above, with high sensitivity by using a trapping agent that traps the chemical substance.

### Underlying Knowledge Forming Basis of the Present Disclosure

International Publication Nos. WO 2019/244464, WO 2018/169022, and WO 2018/169023 and Non-patent Literature 1 describe the detection of a chemical substance using pellet-like organic salt crystals. However, studies by the present inventors show that this method does not necessarily ensure a sufficient contact area with a chemical substance and is less likely to detect the chemical substance with high sensitivity. Furthermore, organic salt crystals collapse or scatter easily by impact, contact, or the like and have poor wearability on a living body, such as a human body, and fixability to an object. Thus, it is difficult to simply detect a chemical substance.

In view of these problems, the present inventors have arrived at a functional member that holds a trapping agent in a porous member. A functional member according to the present disclosure has a structure that holds a trapping agent in a cavity of a porous member. In this structure, an infinite number of cavities of the porous member hold the trapping agent with a particle size small enough to be held in each cavity. Thus, the trapping agent can have a large surface area and improve the detection sensitivity of a chemical substance. Furthermore, the porous member used as a holding substrate can improve wearability on a living body, such as a human body, and fixability to an object, protect the trapping agent from impact, contact, or the like, and prevent the trapping agent from collapsing or being scattered. In other words, a functional member according to the present disclosure is resistant to a mechanical stimulus, such as impact or contact. Thus, a functional member according to the present disclosure can easily detect a chemical substance with high sensitivity.

### Outline of One Aspect of the Present Disclosure

A functional member according to a first aspect of the present disclosure includes:

a porous member with a cavity; and

a trapping agent that is held in the cavity and that traps a chemical substance.

The first aspect can provide a functional member that can easily detect a chemical substance with high sensitivity.

In a second aspect of the present disclosure, for example, in the functional member according to the first aspect, the trapping agent may have an average particle size of 1 μm or less. This can increase the surface area of the trapping agent in the functional member and improve the detection sensitivity of a chemical substance.

In a third aspect of the present disclosure, for example, in the functional member according to the first or second aspect, the cavity may have a pore size of 1 μm or less. This can decrease the particle size of the trapping agent held in the cavity and thereby increase the surface area of the trapping agent in the functional member. The increase in surface area improves the detection sensitivity of a chemical substance in the functional member.

In a fourth aspect of the present disclosure, for example, in the functional member according to any one of the first to third aspects, the porous member may have a porosity of 30% or more. This can efficiently diffuse a chemical substance to be detected into the functional member, thereby increase the probability of trapping the chemical substance by the trapping agent, and improve the detection sensitivity of the chemical substance in the functional member.

In a fifth aspect of the present disclosure, for example, in the functional member according to any one of the first to fourth aspects, the trapping agent in a state of trapping the chemical substance may emit fluorescence characteristic of the state upon irradiation with excitation light. In the fifth aspect, the chemical substance can be detected by an optical method, and the trapped chemical substance can be detected, for example, without contacting the functional member.

In a sixth aspect of the present disclosure, for example, in the functional member according to the fifth aspect, the excitation light may be ultraviolet light.

In a seventh aspect of the present disclosure, for example, in the functional member according to any one of the first to sixth aspects, the trapping agent may be an organic salt.

In an eighth aspect of the present disclosure, for example, in the functional member according to any one of the first to seventh aspects, the chemical substance may contain a hydroxyl radical.

In a ninth aspect of the present disclosure, for example, in the functional member according to the eighth aspect, the trapping agent may be an organic salt containing terephthalic acid and at least one primary alkylamine.

In a tenth aspect of the present disclosure, for example, in the functional member according to any one of the first to ninth aspects, the chemical substance may contain ammonia.

In an eleventh aspect of the present disclosure, for example, in the functional member according to the tenth aspect, the trapping agent may be an organic salt containing a cyanoacrylic acid derivative and a trisubstituted methylamine.

In a twelfth aspect of the present disclosure, for example, in the functional member according to any one of the first to eleventh aspects, the porous member may be a porous sheet with the cavity, and the functional member may be a functional sheet with the trapping agent held in the cavity of the porous sheet. In the twelfth aspect, for example, it is possible to improve the light transmittance of the functional member and/or improve the wearability of the functional member on a living body. Improved light transmittance can contribute to increased sensitivity of a chemical substance in an optical method. Improved wearability on a living body can contribute to prolonged wearing.

In a thirteenth aspect of the present disclosure, for example, in the functional member according to the twelfth aspect, the porous sheet may contain regenerated cellulose. A porous sheet containing regenerated cellulose with a smaller thickness can have strength required for a functional sheet due to the strength improving effect based on hydroxy groups abundantly contained in the regenerated cellulose. A small thickness can contribute to improved light transmittance of the functional member and/or improved wearability of the functional member on a living body.

In a fourteenth aspect of the present disclosure, for example, in the functional member according to the thirteenth aspect, the regenerated cellulose may have a weight-average molecular weight of 150,000 or more. This can increase the number of hydroxy groups per molecule and thereby promote the formation of an intermolecular hydrogen bond. The promoted formation of a hydrogen bond may contribute to the formation of a thin but freestanding functional sheet, for example.

In a fifteenth aspect of the present disclosure, for example, in the functional member according to any one of the twelfth to fourteenth aspects, the functional sheet may have a thickness of 100 nm or more and 2000 nm or less. The functional member according to the fifteenth aspect is particularly suitably worn on a living body by attachment.

In a sixteenth aspect of the present disclosure, for example, in the functional member according to any one of the twelfth to fifteenth aspects, at least one transmittance selected from the group consisting of visible transmittance of the functional sheet and ultraviolet transmittance of the functional sheet may be 10% or more and 90% or less. The sixteenth aspect is particularly suitable for highly sensitive detection of a chemical substance.

In a seventeenth aspect of the present disclosure, for example, in the functional member according to the sixteenth aspect, the at least one transmittance may be 40% or more.

In an eighteenth aspect of the present disclosure, for example, in the functional member according to any one of the twelfth to seventeenth aspects, the functional sheet may be a biocompatible sheet. The eighteenth aspect is particularly suitable for wearing in close contact with a living body.

A chemical substance sensor according to a nineteenth aspect of the present disclosure includes

the functional member according to any one of the first to eighteenth aspects.

The nineteenth aspect can provide a chemical substance sensor that can easily detect a chemical substance with high sensitivity.

In a twentieth aspect of the present disclosure, for example, in the chemical substance sensor according to the nineteenth aspect, the chemical substance sensor may be a living body sensor that detects the chemical substance secreted from a living body.

In a twenty first aspect of the present disclosure, for example, in the chemical substance sensor according to the nineteenth or twentieth aspect, the chemical substance sensor may detect the chemical substance by irradiating the functional member with at least one selected from the group consisting of visible light and ultraviolet light.

In a twenty second aspect of the present disclosure, for example, the chemical substance sensor according to any one of the nineteenth to the twenty first aspects may further include a case that houses the functional member, wherein the case may include a flow path that is located between an outside of the case and the functional member housed in the case, and through a fluid containing the chemical substance may flow through the flow path.

In a twenty third aspect of the present disclosure, for example, in the chemical substance sensor according to the twenty second aspect, the case may include a first member and a second member, and at least one selected from the group consisting of the first member and the second member may include a mechanism for fixing the first member and the second member to each other while the functional member is housed between the first member and the second member.

In the twenty fourth aspect of the present disclosure, for example, in the chemical substance sensor according to the twenty third aspect, the mechanism may fix the first member and the second member to each other by magnetic force of a magnet.

### Embodiments of the Present Disclosure

Embodiments of the present disclosure are described below with reference to the accompanying drawings. The following embodiments are general or specific examples. The numerical values, shapes, materials, constituents, arrangement and connection of the constituents, steps, the sequential order of steps, and the like in the following embodiments are only examples and are not intended to limit the present disclosure. These embodiments can be combined as long as no contradiction arises. Among the constituents in the following embodiments, constituents not described in the independent claims defining the highest level concepts should not be understood as essential constituents. In the following description, like components with substantially the same function are denoted by like reference numerals and may not be further described. Furthermore, to prevent excessively complex drawings, some elements may not be shown.

### [Functional Member]

FIG. 1 illustrates an example of a functional member according to the present disclosure. The functional member in FIG. 1 is a functional sheet 1 including a porous sheet 2, which is a porous member, and a trapping agent 3, which traps a chemical substance. The trapping agent 3 is held in a cavity 4 of the porous sheet 2. In FIG. 1, the cavity 4 and the trapping agent 3 held in the cavity 4 are illustrated in an enlarged region R1 of the functional sheet 1. The shape of the cavity 4 and the state of the trapping agent 3 in the cavity 4 in FIG. 1 are only schematic. The actual shape and state are not limited to those illustrated in FIG. 1.

The porous sheet 2 can function as a substrate of the functional sheet 1. The porous sheet 2 includes a plurality of cavities 4.

Examples of a material constituting the porous sheet 2 include polymers, metals, metal compounds, and composite materials thereof. The polymers include natural polymers, semi-synthetic polymers, and synthetic polymers. Examples of the natural polymers include cellulose. Examples of the semi-synthetic polymers include regenerated cellulose, chemically modified cellulose, and cellulose derivatives, such as methylcellulose, carboxymethylcellulose, and cellulose acetate. Examples of the synthetic polymers include polyolefins, such as polyethylene and polypropylene, polyesters, such as poly(ethylene terephthalate) and poly(ethylene naphthalate), acrylics, such as polyacrylonitrile, poly(vinyl alcohol) and its derivatives, polyurethanes, and fluoropolymers, such as polytetrafluoroethylene (PTFE), poly(vinylidene difluoride) (PVDF), and ethylene-tetrafluoroethylene copolymers (ETFE). Examples of the metals include titanium, aluminum, and stainless steel. Examples of the metal compounds include metal oxides. Examples of the metal oxides include alumina. However, a material constituting the porous sheet 2 is not limited to these examples.

The porous sheet 2 may contain at least one material selected from the above material groups as a main component. The term “main component”, as used herein, refers to a component with the highest content expressed in % by weight. The main component content is, for example, 50% or more by weight and may be 60% or more by weight, 70% or more by weight, 80% or more by weight, 90% or more by weight, or 95% or more by weight. The porous sheet 2 may be composed of the at least one material.

The porous sheet 2 may contain fiber of the at least one material or may be composed of the fiber. The fiber may be a composite fiber of two or more materials. Examples of the porous sheet 2 containing fiber include paper, woven fabric, and nonwoven fabric. The porous sheet 2 may be a stretched porous film of a fluoropolymer, for example, a PTFE expanded porous film, which is also referred to as ePTFE. A stretched porous film of a fluoropolymer has a characteristic porous structure containing many fine fibrils made of the fluoropolymer and many cavities between the fibrils. This porous structure is different from the structure of paper, woven fabric, and nonwoven fabric. A stretched porous film of a fluoropolymer can be the porous sheet 2 having the cavities 4 with a smaller pore size. However, the porous sheet 2 is not limited to these examples as long as the porous sheet 2 has a plurality of cavities 4.

The porous sheet 2 may contain regenerated cellulose. The porous sheet 2 containing regenerated cellulose with a smaller thickness can have strength required for the functional sheet 1 due to the strength improving effect based on hydroxy groups abundantly contained in the regenerated cellulose. The small thickness results in, for example, the functional sheet 1 with improved light transmittance and can contribute to highly sensitive detection of a chemical substance by an optical method. A sheet with a small thickness can contribute to improved wearability on a living body. The functional sheet 1 with improved wearability is particularly suitable, for example, for wearing in close contact with a living body, such as a human body, and prolonged wearing. The porous sheet 2 may be composed of regenerated cellulose. When the porous sheet 2 containing regenerated cellulose has a regenerated cellulose content of 80% or more by weight, the density of hydrogen bonds formed by hydroxy groups of the regenerated cellulose increases, and the effect of improving the strength of the porous sheet 2 and the functional sheet 1 becomes more reliable. Furthermore, the improved strength can contribute to improved handleability of the porous sheet 2 and the functional sheet 1.

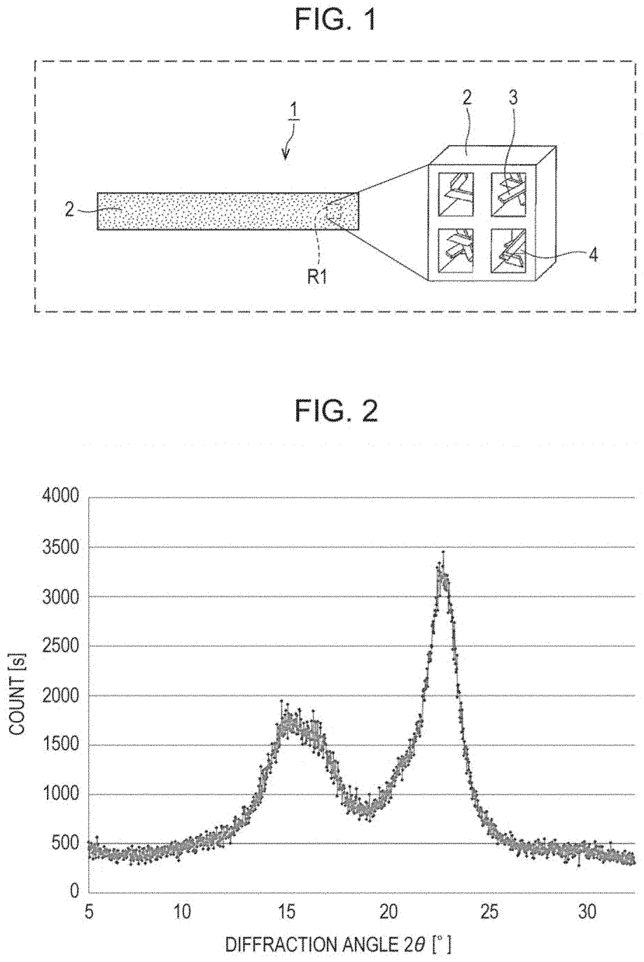

Cellulose includes natural cellulose and regenerated cellulose. The term “regenerated cellulose”, as used herein, refers to cellulose without a crystal structure I characteristic of natural cellulose. The crystal structure of cellulose can be determined by wide-angle X-ray diffraction (hereinafter referred to as XRD). FIG. 2 shows an XRD pattern of natural cellulose. The pattern shown in FIG. 2 was measured using CuKα radiation generated at a voltage of 50 kV and at an electric current of 300 mA as X-rays. The pattern shown in FIG. 2 has peaks at diffraction angles in the range of approximately 14 to 17 degrees and 23 degrees, which correspond to the crystal structure I. The phrase “cellulose without a crystal structure I”, as used herein, refers to cellulose without distinct peak tops at diffraction angles in the range of 14 to 17 degrees and 23 degrees. Regenerated cellulose typically has a crystal structure II. Thus, an XRD pattern of regenerated cellulose has no peaks at diffraction angles in the range of approximately 14 to 17 degrees and 23 degrees, which correspond to the crystal structure I, but has peaks at diffraction angles of approximately 12 degrees, 20 degrees, and 22 degrees, which correspond to the crystal structure II.

In general, regenerated cellulose substantially has a molecular structure represented by the following formula (1). The formula (1) shows a linear molecular structure with a glucose unit as a repeating unit. The phrase “substantially has”, as used herein, means that the regenerated cellulose is not limited to a form strictly having the molecular structure represented by the formula (1) and may have a change in the glucose unit and the molecular structure of the regenerated cellulose. For example, the hydroxy groups of the glucose unit may be partly converted to another group by derivatization or chemical modification. With respect to the allowable degree of conversion, for example, the ratio of the number of hydroxy groups actually maintained to the number of hydroxy groups when all the hydroxy groups in the molecular structure of the formula (1) are not converted to another group, in other words, when all the hydroxy groups are maintained may be 90% or more, 95% or more, or 98% or more. The ratio can be determined by a known method, such as X-ray photoelectron spectroscopy (XPS). Regenerated cellulose may have a branched molecular structure.

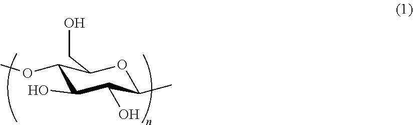

As understood from the formula (1), the porous sheet 2 containing regenerated cellulose contains abundant hydroxy groups. A hydrogen bond is formed between hydroxy groups. A hydrogen bond is formed not only in the molecule but also between molecules of regenerated cellulose. Thus, the porous sheet 2 containing regenerated cellulose and the functional sheet 1 including the porous sheet 2 may have high strength due to many hydrogen bonds.

Regenerated cellulose may not be cross-linked. Regenerated cellulose does not include artificially derivatized cellulose. However, regenerated cellulose includes cellulose that is once derivatized and is then regenerated.

Regenerated cellulose may have a weight-average molecular weight of 150,000 or more, 180,000 or more, or 200,000 or more. This can increase the number of hydroxy groups per molecule and thereby promote the formation of an intermolecular hydrogen bond. In such a case, even when the porous sheet 2 containing regenerated cellulose is a thin sheet with a thickness of, for example, 100 nm or more and 2000 nm or less, a freestanding sheet can be formed more reliably. The freestanding porous sheet 2 and the functional sheet 1 including the freestanding porous sheet 2 as a substrate can be prevented from being torn, for example, when attached to a living body, such as a human body. The weight-average molecular weight of regenerated cellulose can be determined by gel permeation chromatography (hereinafter referred to as GPC). The term “freestanding sheet”, as used herein, refers to a sheet that can maintain its own shape without a support. For example, when a portion of a freestanding sheet is gripped and held in the air with fingers or tweezers, the sheet can have sufficient strength to resist the breakage of the portion or another portion of the sheet.

The viscosity of a solution containing regenerated cellulose generally increases with the weight-average molecular weight of the regenerated cellulose. The porous sheet 2 and the functional sheet 1 containing regenerated cellulose can be formed from a solution containing the regenerated cellulose. However, these sheets are difficult to form from a solution with excessively high viscosity. A sheet formed from a solution with appropriate viscosity can have a more uniform thickness. From the above perspective, the upper limit of the weight-average molecular weight of regenerated cellulose is, for example, 1,000,000 or less and may be 500,000 or less.

Examples of raw materials of regenerated cellulose include cellulose derived from plants, such as pulp and cotton, and cellulose produced by microorganisms, such as bacteria. The raw materials of regenerated cellulose are not limited to these examples. The impurity concentration of raw materials may be 20% or less by weight.

Regenerated cellulose typically has a high affinity for both hydrophilic and hydrophobic materials. Thus, the porous sheet 2 containing regenerated cellulose is particularly suitable for holding both a hydrophilic trapping agent 3 and a hydrophobic trapping agent 3.

The porous sheet 2 may be subjected to various treatments, such as hydrophilization treatment. A hydrophilized porous sheet 2 can have improved wearability on a human body, for example. The hydrophilization treatment can be performed by a known method.

The porous sheet 2 may contain a hydrophilic material. This can improve wearability on a human body, for example. Examples of the hydrophilic material include regenerated cellulose, hydrophilized PTFE, and hydrophilized PVDF.

The porous sheet 2 may contain another material, such as a ceramic or an additive agent. The porous sheet 2 containing regenerated cellulose may contain incidental impurities of a method for producing the regenerated cellulose.

The porous sheet 2 may be a filter, such as a filter paper, a membrane filter, or a depth filter. The filter may be composed of fibers. Examples of fibers constituting the filter include glass fibers and cellulose fibers. When the fibers constituting the filter are cellulose fibers, the porous sheet 2 and the functional sheet 1 have improved flexibility.

The cavities 4 in the porous sheet 2 may have a pore size of, for example, 1 μm or less and may be 0.8 μm or less, 0.6 μm or less, 0.5 μm or less, 0.3 μm or less, 0.2 μm or less, or 0.1 μm or less. The lower limit of the pore size is, for example, 0.1 nm or more and may be 1 nm or more or 2 nm or more. The pore size may be 0.1 nm or more and 800 nm or less, or 1 nm or more and 100 nm or less. The cavities 4 with a smaller pore size can result in the trapping agent 3 with a smaller particle size held in the cavities 4 and the trapping agent 3 with an increased surface area in the functional sheet 1. The increase in surface area improves the detection sensitivity of a chemical substance in the functional sheet 1. The functional sheet 1 having the cavities 4 with a pore size in the above range is suitable, for example, for detecting a trace amount of chemical substance, such as chemical substance secreted from a living body, such as a human body. Depending on the application of the functional sheet 1, however, the cavities 4 may have a pore size beyond the above range. The pore size of the cavities 4 is typically larger than the average particle size of the trapping agent 3 in the functional sheet 1.

The pore size of the cavities 4 may be equal to or less than the wavelength of visible light and in some cases may be equal to or less than the wavelength of visible light and ultraviolet light. This can reduce the scattering of the light in the cavities 4 and thereby improve the light transmittance of the functional sheet 1. Improved light transmittance can result in an optical method with further improved detection sensitivity and an inconspicuous sheet when attached to a living body, such as a human body.

The pore size of the cavities 4 can be determined, for example, in pore distribution measurement using a mercury intrusion method or a gas adsorption method. More specifically, the peak pore size in a Log differential pore volume distribution plot measured by a BJH method can be the pore size of the cavities 4. For example, the pore size can be determined using the following equation from a bubble point pressure measured by a bubble point method defined in Japan Industrial Standard K 3832. In the following equation, the unit of the pore size d is meter (m), γ denotes the surface tension (unit: N/m) of a solvent used to measure the bubble point pressure, θ denotes the contact angle (unit: degree) of the solvent on a material constituting the porous sheet, and ΔP denotes the bubble point pressure (unit: Pa). For a hydrophilic porous sheet 2, pure water can be used as a solvent. For a hydrophobic porous sheet 2, a liquid mixture of pure water and an alcohol can be used as the solvent. Examples of the alcohol include ethanol and isopropyl alcohol.

Pore size d=(4·γ·cos θ)/ΔP Equation:

The porosity of the porous sheet 2, which is the occupancy of the cavities in the porous sheet 2, is, for example, 30% or more and may be 40% or more, 50% or more, 60% or more, or 70% or more. The upper limit of the porosity is, for example, 99% or less. At a higher porosity, a chemical substance to be detected diffuses more efficiently into the functional sheet 1. This can increase the probability of trapping the chemical substance by the trapping agent 3 and improve the detection sensitivity of the chemical substance in the functional sheet 1. The functional sheet 1 with a porosity in the above range is suitable, for example, for detecting a trace amount of chemical substance, such as chemical substance secreted from a living body, such as a human body. Depending on the application of the functional sheet 1, however, the porosity may be below the above range.

The porosity of the porous sheet 2 can be calculated by substituting the weight, thickness, and area (main surface area) of the sheet and the true density of the material constituting the sheet into the following equation.

Porosity (%)={1−(weight [g]/(thickness [cm]×area [cm²]×true density [g/cm³]))}×100

The porous sheet 2 may have a pore size and a porosity in the above range, for example, a pore size of 1 μm or less and a porosity of 30% or more. This can particularly improve the detection sensitivity of a chemical substance.

The thickness of the porous sheet 2 is, for example, in the range of 0.1 μm to 1000 μm and may be in the range of 30 μm to 230 μm. In consideration of adhesiveness to the skin of a living body, such as a human body, in particular, the porous sheet 2 containing regenerated cellulose may have a thickness of 100 nm or more and 2000 nm or less, 300 nm or more and 1300 nm or less, or 300 nm or more and 1000 nm or less. However, the thickness of the porous sheet 2 is not limited to these examples. The thickness of the porous sheet 2 may vary depending on the application and the specific use of the functional sheet 1.

The shape of the porous sheet 2, for example, viewed perpendicularly to the main surface of the sheet is a polygon including a square or a rectangle, a circle including an approximate circle, an ellipse including an approximate ellipse, a belt-like shape, or an indefinite shape. The polygon may have a rounded corner. However, the shape of the porous sheet 2 is not limited to these examples.

The trapping agent 3 has the function of trapping a chemical substance. Examples of the chemical substance include a hydroxyl radical and ammonia. The trapping agent 3 may trap a hydroxyl radical in the gas or ammonia in the gas. The chemical substance may be a gas species or a liquid species secreted from a living body, such as a human body. The chemical substance may be a metabolite of a living body. It is known that hydroxyl radicals and ammonia are secreted from a living body and that stress increases the amount of hydroxyl radicals and ammonia produced in the living body. Examples of the liquid species include sodium, potassium, calcium, chlorine, sodium chloride, and lactic acid contained in sweat or body fluids. It is known that fatigue increases the amount of lactic acid produced in a living body. However, the chemical substance is not limited to these examples. The functional sheet 1 can trap various chemical substances depending on the type of the trapping agent 3.

An example of the trapping agent 3 is an organic salt. The organic salt contains, for example, an organic acid as an anion and a protonated base as a cation. Examples of the organic acid include carboxylic acids and sulfonic acids. Examples of the base include amines. However, the organic salt, organic acid, and base are not limited to these examples. An organic acid and a base are usually bonded together by an ionic bond. The organic salt may be a crystalline organic salt with a crystal structure. The crystal structure may be composed of an organic acid and a base. The crystal structure may be a supramolecular crystal structure containing an organic acid molecule and a base molecule.

In such a case, the organic salt is a supramolecular crystal. The term “supramolecular”, as used herein, refers to a regular array structure formed by noncovalent bonding of two or more types of molecules. Examples of the noncovalent bonding are ionic bonding, hydrogen bonding, and π-π interaction.

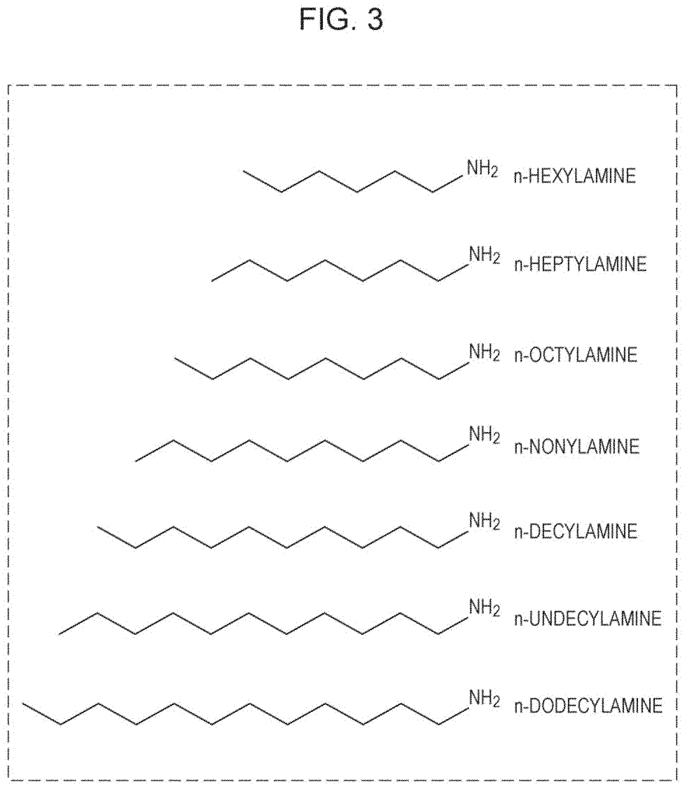

An example of the organic salt is an organic salt A containing terephthalic acid and at least one primary alkylamine. An alkyl group constituting the primary alkylamine has 6 or more and 17 or less carbon atoms, for example. The number of carbon atoms in the alkyl group may be 8 or more and may be 12 or less. Examples of the primary alkylamine include n-hexylamine, n-heptylamine, n-octylamine, n-nonylamine, n-decylamine, n-undecylamine, and n-dodecylamine illustrated in FIG. 3. The organic salt A may have a supramolecular crystal structure containing a primary alkylamine molecule and a terephthalic acid molecule. The organic salt A may have a space between a primary alkylamine molecule and a terephthalic acid molecule. The organic salt A can trap a hydroxyl radical. A hydroxyl radical is trapped, for example, in a space between a primary alkylamine molecule and a terephthalic acid molecule. The organic salt A that has trapped a hydroxyl radical contains hydroxy terephthalic acid and at least one primary alkylamine. The hydroxy terephthalic acid is formed from terephthalic acid and the trapped hydroxyl radical by the reaction represented by the following formula. Terephthalic acid and hydroxy terephthalic acid have different characteristics of fluorescence emitted by ultraviolet irradiation. In the organic salt A, due to the different characteristics, a hydroxyl radical can be detected by an optical method. The organic salt A may be an organic salt disclosed in International Publication No. WO 2019/244464.

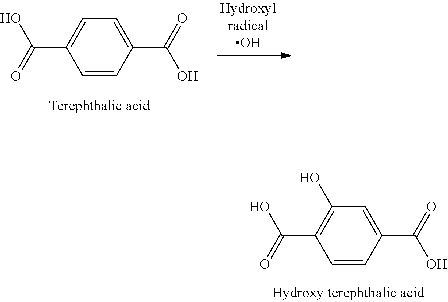 [SMILES: O=C(O)c1ccc(C(=O)O)cc1 ; O=C(O)c1ccc(C(=O)O)c(O)c1]

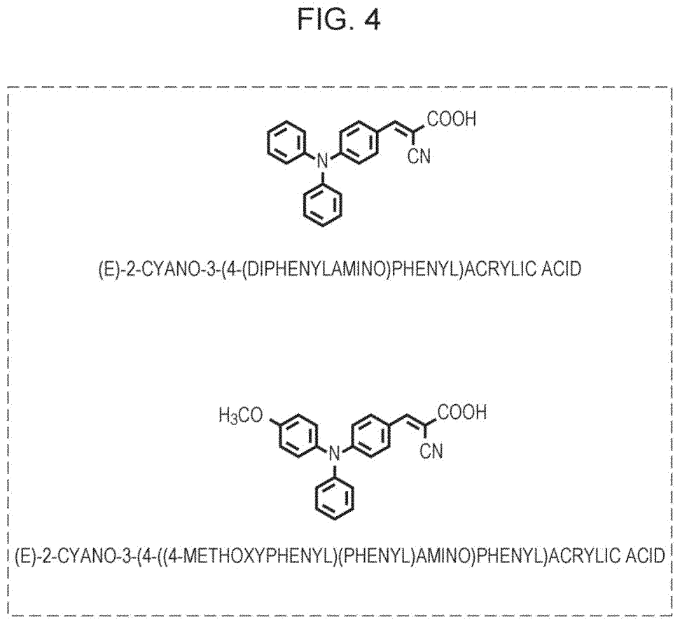

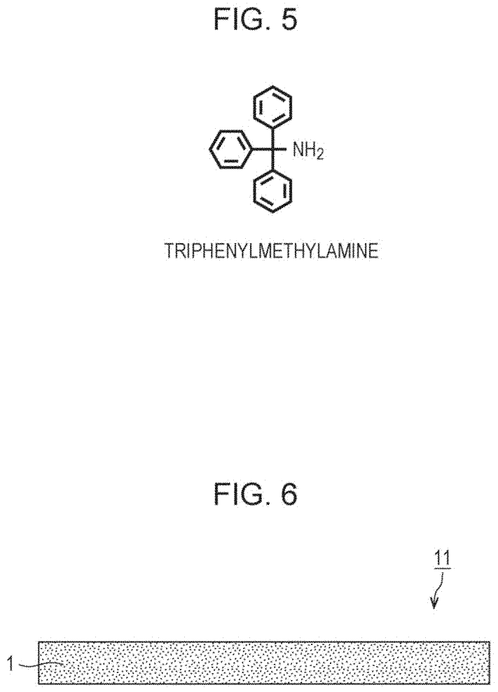

Another example of the organic salt is a crystalline organic salt B containing a cyanoacrylic acid derivative and a trisubstituted methylamine. The organic salt B has a structure in which supramolecular units composed of two or more types of molecules are arranged, and the supramolecular units may be a complex crystal containing a cyanoacrylic acid derivative and a trisubstituted methylamine as the molecules. The complex crystal may have a molecular cavity, which has no guest molecule for a supramolecular unit host, between the supramolecular units. The complex crystal may have a supramolecular unit in which the bonding ratio of the cyanoacrylic acid derivative to the trisubstituted methylamine is not 4:4. Examples of the cyanoacrylic acid derivative are illustrated in FIG. 4. The examples in FIG. 4 are (E)-2-cyano-3-(4-(diphenylamino)phenyl)acrylic acid and (E)-2-cyano-3-(4-((4-methoxyphenyl)(phenyl)amino)phenyl)acrylic acid. An example of the trisubstituted methylamine is illustrated in FIG. 5. The example in FIG. 5 is triphenylmethylamine. The organic salt B can trap ammonia. For example, ammonia is physically adsorbed inside the organic salt B. The organic salt B may be a complex crystal disclosed in International Publication Nos. WO 2018/169022 or WO 2018/169023.

The trapping agent 3, which is an organic salt, is not limited to these examples.

Other examples of the trapping agent 3 include cyclodextrins that can trap at least one selected from the group consisting of inorganic metals, such as Na and Ka, and organic materials, and antibodies and enzymes that can trap a specific chemical substance. An example of the enzymes is an oxidoreductase of lactic acid. For example, the enzyme can detect lactic acid secreted from a living body.

The trapping agent 3 is not limited to these examples.

The average particle size of the trapping agent 3 is, for example, 1 μm or less and may be less than 1 μm, 0.8 μm or less, 0.6 μm or less, 0.5 μm or less, 0.3 μm or less, or 0.2 μm or less. The lower limit of the average particle size is, for example, 0.1 nm or more and may be 1 nm or more or 2 nm or more. An average particle size in the above range can result in the trapping agent 3 with an increased surface area in the functional sheet 1 and the functional sheet 1 with improved detection sensitivity of a chemical substance. The functional sheet 1 containing the trapping agent 3 with an average particle size in the above range is suitable, for example, for detecting a trace amount of chemical substance, such as chemical substance secreted from a living body, such as a human body. Depending on the application of the functional sheet 1, however, the trapping agent 3 may have an average particle size beyond the above range. The average particle size of the trapping agent 3 in the functional sheet 1 is typically smaller than the pore size of the cavities 4.

The average particle size of the trapping agent 3 can be an average value of at least 20 particle sizes of the trapping agents 3 determined by acquiring an enlarged observation image of at least one surface selected from the surfaces and cross sections of the functional sheet 1 by a magnification observation method, such as with a scanning electron microscope (SEM), and analyzing the acquired image. The particle size of the trapping agent 3 is defined as the diameter of a circle with the same area as a particle observed on an enlarged observation image. An image processing method may be used for the analysis.

The trapping agent 3 in a state of trapping a chemical substance may emit fluorescence characteristic of the state upon excitation light irradiation. In such a case, a chemical substance can be detected by an optical method by detecting fluorescence emitted from the trapping agent 3. Furthermore, for example, it is also possible to detect a chemical substance trapped by the functional sheet 1 without contacting the functional sheet 1. Depending on the types of the trapping agent 3 and chemical substance, it is also possible to quantitatively evaluate the trapped chemical substance by detecting the intensity of emitted fluorescence. Furthermore, the functional sheet 1 with high excitation light transmittance and fluorescence transmittance can detect the emission and fluorescence of the light through a surface opposite to the surface exposed to a chemical substance and can therefore detect the chemical substance, for example, in the state where the functional sheet 1 is attached to a living body, such as a human body. The trapping agent 3 that emits the characteristic fluorescence includes a trapping agent that does not fluoresce before trapping a chemical substance but fluoresces after trapping the chemical substance and a trapping agent that emits different types of fluorescence before and after trapping a chemical substance. Excitation light applied to the trapping agent 3 may be light with a wavelength of 200 nm or more and 800 nm or less, ultraviolet light with a wavelength of 200 nm or more and less than 400 nm, or visible light with a wavelength of 400 nm or more and 800 nm or less. The trapping agent 3 may emit different fluorescence for each trapped chemical substance. The fluorescence may be light with a wavelength of 200 nm or more and 800 nm or less, ultraviolet light with a wavelength of 200 nm or more and less than 400 nm, or visible light with a wavelength of 400 nm or more and 800 nm or less.

Examples of the trapping agent 3 that emits the characteristic fluorescence include the organic salt A and the organic salt B. The organic salt A that has trapped a hydroxyl radical emits fluorescence with a peak in the wavelength range of 412 to 435 nm upon light irradiation at a wavelength of approximately 310 nm. The organic salt A enables the quantitative determination of a trapped hydroxyl radical from a change in fluorescence intensity. The organic salt B that has trapped ammonia emits fluorescence with a peak of approximately 525 nm upon light irradiation at a wavelength of 365 nm. The organic salt B enables the quantitative determination of trapped ammonia from a change in fluorescence intensity.

The trapping agent 3 may be held near the center of the thickness of the porous sheet 2 or the functional sheet 1 or may be held near a surface of the porous sheet 2 or the functional sheet 1. The trapping agent 3 may be held throughout the porous sheet 2 or the functional sheet 1 or may be held uniformly throughout the porous sheet 2 or the functional sheet 1.

Retention of the trapping agent 3 in the functional sheet 1 can be confirmed by the following method, for example. The method A is an example in which a crystalline material is used as the trapping agent 3.

### Method A: XRD

The functional sheet 1 is subjected to XRD. When a diffraction peak derived from the trapping agent 3 appears in an XRD pattern, it can be judged that the trapping agent 3 is held in the functional sheet 1.

### Method B: Magnification Observation Method Using an Electron Microscope or the Like

An enlarged observation image is acquired for at least one surface selected from the group consisting of the surfaces and cross sections of the functional sheet 1. An example of the enlarged observation image is an image acquired with an electron microscope, such as SEM. Whether the trapping agent 3 is held in the cavities 4 of the porous sheet 2 can be confirmed by analyzing the enlarged observation image. An image processing method may be used for the analysis.

The method A and the method B may be used in combination.

At least one light transmittance selected from the group consisting of the visible transmittance T_{V} of the functional sheet 1 and the ultraviolet transmittance T_{UV} of the functional sheet 1 may be 10% or more and 90% or less. When the at least one light transmittance of the functional sheet 1 is 10% or more, the size of the trapping agent 3 held is small enough to reduce the scattering of at least one selected from the group consisting of visible light and ultraviolet light. Furthermore, the detection of a trapped chemical substance by an optical method, such as the detection of excitation light emission and fluorescence, can reduce optical loss in the detection. Thus, the functional sheet 1 is particularly suitable for highly sensitive detection of a chemical substance. Furthermore, a visible transmittance T_{V} of 10% or more can advantageously result in an inconspicuous sheet when attached to a living body, such as a human body. The at least one light transmittance may be 20% or more, 30% or more, or 40% or more. This advantage becomes more significant as the light transmittance increases.

The visible transmittance T_{V} refers to the luminous transmittance of a visible portion defined in JIS T8141: 2016. The spectral transmittance is measured with a spectrophotometer for light with a wavelength of 400 nm or more and 800 nm or less. The visible transmittance can be roughly determined by comparison with a limit sample with a known visible transmittance.

The ultraviolet transmittance T_{UV} refers to an ultraviolet transmittance defined in JIS T8141: 2016. The wavelength of light to be evaluated is 300 nm, 310 nm, or 365 nm. The ultraviolet transmittance T_{UV} may be determined by measuring ultraviolet transmittance at a plurality of wavelengths, for example, 300 nm, 310 nm, and 365 nm and averaging the measured transmittance values.

When the trapping agent 3 in a state of trapping a chemical substance emits fluorescence characteristic of the state upon excitation light irradiation, the functional sheet 1 may have a transmittance of 10% or more and 90% or less at the wavelength of the excitation light and at the wavelength of the fluorescence. This can reduce optical loss in the detection of excitation light emission and fluorescence. In such a case, fluorescence generated by excitation light irradiation from one surface of the functional sheet 1 may be detected from the other surface of the functional sheet 1. The transmittance may be 20% or more, 30% or more, or 40% or more.

The thickness of the functional sheet 1 is, for example, in the range of 0.1 μm to 1000 μm and may be in the range of 30 μm to 230 μm. In consideration of adhesiveness to the skin of a living body, such as a human body, in particular, the functional sheet 1 including the porous sheet 2 containing regenerated cellulose may have a thickness of 100 nm or more and 2000 nm or less, 300 nm or more and 1300 nm or less, or 300 nm or more and 1000 nm or less. The functional sheet 1 with a thickness of 100 nm or more can more reliably have the freestanding property. The functional sheet 1 with a thickness of 300 nm or more can hold a larger amount of trapping agent 3, for example. The functional sheet 1 containing regenerated cellulose with a thickness of 2000 nm or less can be attached to the human skin without an adhesive agent, for example. This is because, in addition to adhesiveness resulting from van der Waals force due to the small thickness, regenerated cellulose rich in hydroxy groups has high adhesiveness to the skin. An adhesive agent may cause rough skin, rash, stuffiness, allergies, and the like. Attachment to the skin without an adhesive agent is therefore very advantageous. A thickness of 1300 nm or less is particularly suitable for long-term stable attachment to the skin without an adhesive agent. The functional sheet 1 with a thickness of 1000 nm or less attached to the skin is rarely noticed by a third person. However, the thickness of the functional sheet 1 is not limited to these examples. The thickness of the functional sheet 1 may vary depending on the application and the specific use.

The thickness of the functional sheet 1 is determined as an average value of the thicknesses measured at at least five measurement points. The thickness of the functional sheet 1 can be measured with a profiler, for example.

The shape of the functional sheet 1, for example, viewed perpendicularly to the main surface of the sheet is a polygon including a square or a rectangle, a circle including an approximate circle, an ellipse including an approximate ellipse, a belt-like shape, or an indefinite shape. The polygon may have a rounded corner. However, the shape of the functional sheet 1 is not limited to these examples. The functional sheet 1 may have the same shape as the porous sheet 2.

The functional sheet 1 may have an area of 7 mm² or more when attached to a living body or the like. The area may be 100 mm² or more and 1735 mm² or less. Depending on the application, however, the functional sheet 1 may have an area outside the above range.

The functional sheet 1 may be a biocompatible sheet. The term “biocompatible”, as used herein, refers to the property that rarely causes a reaction, such as eruption or inflammation, when attached to a living body, particularly to the skin. Biocompatibility can be evaluated by a human patch test, for example.

The functional sheet 1 may include two or more porous sheets 2. The functional sheet 1 may include any layer or member other than the porous sheet 2. For detection with higher sensitivity, however, the functional sheet 1 may be composed of one or two or more porous sheets 2 or may be composed of one porous sheet 2.

The functional sheet 1 can be used as a chemical substance detection sheet, for example. The functional sheet 1 can also be used to produce a chemical substance sensor for detecting a chemical substance. Chemical substance sensors are also referred to as chemosensors. The functional sheet 1 can be placed to face a space, such as the interior of a room, for example. Examples of placement surfaces are surfaces of furniture and fixtures, such as desks and shelves, and the surfaces of walls. In such a case, it is possible to detect a chemical substance in the room atmosphere. Depending on the form of the functional sheet 1, such as the type of the trapping agent 3, it is also possible to detect the concentration distribution of a chemical substance in the space. The functional sheet 1 can be placed near a living body, such as a human body, for example. The functional sheet 1 may also be attached to a living body. In such a case, it is possible to detect a chemical substance secreted from the living body. The functional sheet 1 can also be used to produce a living body sensor for detecting a chemical substance secreted from a living body. Examples of placement surfaces on a living body are skin, mucosae, and internal organs. However, the placement surfaces on a living body are not limited to these examples. Furthermore, the application and usage of the functional sheet 1 are not limited to these examples.

The functional sheet 1 may be placed on another substrate. Examples of the other substrate include quartz glass and resin films, such as PET films and cellophane films. When the trapping agent 3 in a state of trapping a chemical substance emits fluorescence characteristic of the state upon excitation light irradiation and when the transmittance of the other substrate is 10% or more at the wavelength of the excitation light and fluorescence, fluorescence emitted from one of the functional sheet 1 and the other substrate by excitation light irradiation may be detected by the other.

The functional member in FIG. 1 is the functional sheet 1 including the porous sheet 2 as a porous member. In other words, the shapes of the porous member and the functional member in FIG. 1 are sheets. However, the shapes of a porous member and a functional member according to the present disclosure are not limited to sheets. A porous member and a functional member with a shape other than sheets can have any combination of the forms and characteristics described above in the description of the porous sheet 2 and the functional sheet 1 without restriction due to the shape. Examples of applications and usage of a functional member with a shape other than sheets are the same as those of the functional sheet 1.

### [Method for Producing Functional Member]

A method for producing a functional member is described below with reference to a method for producing the functional sheet 1. A functional member with a shape other than sheets can also be produced by the same method as the functional sheet 1.

The functional sheet 1 can be produced by the following method, for example. The following method is an example in which an organic salt is used as the trapping agent 3. The method for producing the functional sheet 1 is not limited to the following method.

A solution of an organic salt dissolved in a solvent is prepared. The porous sheet 2 is then brought into contact with the solution. The concentration of the solution is typically equal to or lower than the solubility of the organic salt. The term “solubility”, as used herein, refers to the concentration of a saturated solution. The contact may be performed, for example, by a method of immersing the porous sheet in the solution or by a method of applying the solution to the porous sheet. The immersion may be performed until the cavities 4 of the porous sheet 2 are filled with the solution. The solution can be applied by a coating method, such as spraying, gravure printing, gap coating, or die coating. The solvent is then removed by drying. The removal of the solvent causes the organic salt to precipitate in the cavities 4 of the porous sheet 2, thus forming the functional sheet 1. For the drying, various drying methods, such as natural drying, vacuum drying, heat drying, freeze drying, and supercritical drying, can be utilized. The drying may be performed in combination with heating, for example, vacuum heating. This method can make the distribution of the organic salt in the porous sheet 2 more uniform. Furthermore, an organic salt made into fine particles by a mechanical method, such as pulverization, or held in a powder state in a porous member is easily modified by mechanical stimulation and changes or loses its optical characteristics, such as fluorescence properties. The method described above can suppress the modification.

Depending on the type of the porous sheet 2, the formation of the porous sheet 2 and the holding of the trapping agent 3 in the cavities 4 may be performed simultaneously. The functional sheet 1 including the porous sheet 2 containing regenerated cellulose can also be produced by this method.

The functional sheet 1 including the porous sheet 2 containing regenerated cellulose can be produced by the following method, for example.

First, cellulose is dissolved in a solvent to prepare a cellulose solution. The cellulose may be cellulose derived from a plant, such as pulp or cotton, or cellulose produced by a microorganism, such as bacterium. The cellulose may have a weight-average molecular weight in the range described above. The impurity concentration of the raw material cellulose is desirably 20% or less by weight.

The solvent may be a solvent containing an ionic liquid. However, the solvent is not limited to this example as long as the solvent can dissolve the cellulose. A solvent containing an ionic liquid can dissolve the cellulose in a relatively short time. The ionic liquid is a salt composed of an anion and a cation. The ionic liquid is in a liquid state at a temperature of 150° C. or less, for example. An example of the ionic liquid is an ionic liquid C containing at least one selected from the group consisting of amino acids and alkyl phosphates. The use of a solvent containing the ionic liquid C can reduce the decrease in the molecular weight of the cellulose. Amino acids are components present in a living body, and the use of a solvent containing the ionic liquid C can therefore improve the biocompatibility of the functional sheet 1.

An example of the ionic liquid is represented by the following formula (s1). An ionic liquid D represented by the formula (s1) is an example of the ionic liquid C. The anion of the ionic liquid D is an amino acid. As shown in the formula (s1), the anion of the ionic liquid D has a terminal carboxy group and a terminal amino group. The cation of the ionic liquid D may be a quaternary ammonium cation.

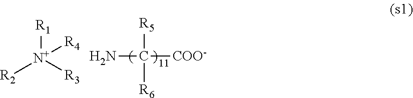

R₁ to R₆ in the formula (s1) independently denote a hydrogen atom or a substituent. The substituent is an alkyl group, a hydroxyalkyl group, or a phenyl group. The carbon chain of the substituent may have a branch. The substituent may have at least one group selected from the group consisting of an amino group, a hydroxy group, and a carboxy group. n denotes an integer of 1 or more and 5 or less.

Another example of the ionic liquid is represented by the following formula (s2). An ionic liquid E represented by the formula (s2) is an example of the ionic liquid C. The anion of the ionic liquid E is an alkyl phosphate.

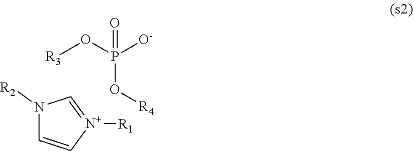

R₁ to R₄ in the formula (s2) independently denote a hydrogen atom or an alkyl group having 1 to 4 carbon atoms.

The cellulose solution is then applied to a substrate to form a liquid film, and the liquid film is gelled to form a polymer gel sheet supported by the substrate. The liquid film can be formed by a coating method, such as gap coating, slot die coating, spin coating, coating with a bar coater, knife coating, or gravure coating. The gelation can be performed, for example, by bringing the liquid film into contact with a rinse that does not dissolve cellulose. The contact with the rinse removes the ionic liquid from the liquid film and forms a polymer gel sheet. The rinse may be brought into contact with the liquid film by immersing the substrate and the liquid film in the rinse. The contact with the rinse may be performed multiple times. This step is also the step of washing the polymer gel sheet.

The rinse is, for example, a solvent that does not dissolve cellulose and that is compatible with the ionic liquid. Examples of the solvent include water, methanol, ethanol, propanol, butanol, octanol, toluene, xylene, acetone, acetonitrile, dimethylacetamide, dimethylformamide, and dimethyl sulfoxide.

The trapping agent 3 is then introduced into the polymer gel sheet. For example, the trapping agent can be introduced by bringing a solution containing the trapping agent into contact with the polymer gel sheet. The trapping agent is dissolved or dispersed in the solution to be contacted. The polymer gel sheet may be brought into contact with the solution by immersing the polymer gel sheet in the solution. In the immersion, ultrasonic waves may be applied to the polymer gel sheet in ultrasonication. The ultrasonication further ensures the inclusion of the trapping agent in the polymer gel sheet and further ensures the formation of the porous sheet 2 with fine cavities. For the ultrasonication, a sound wave with a frequency of 10 kHz or more can be used. It is desirable to perform the ultrasonication for 5 seconds or more. The polymer gel sheet may be brought into contact with the solution by applying the solution to the polymer gel sheet. The solution can be applied by a coating method, such as spraying, gravure printing, gap coating, or die coating.

In the immersion, at least one selected from the group consisting of shaking and liquid flow may be applied to the solution. In such a case, the trapping agent 3 can be more uniformly and finely contained. It is desirable that the shaking cycle be 5 rpm or more. It is desirable that the flow rate of the liquid flow be 1 mL/min or more.

The ultrasonication, the process of applying shaking, and the process of applying liquid flow may be performed during the immersion in the rinse.

Unnecessary components, such as the rinse and the solvent, are then removed from the polymer gel sheet to prepare the functional sheet 1. The porous sheet 2 is formed from the polymer gel sheet by removing the unnecessary components. The trapping agent 3 may be precipitated inside the porous sheet 2 by removing the unnecessary components. The removal can be performed by drying, for example. For the drying, various drying methods, such as natural drying, vacuum drying, heat drying, freeze drying, and supercritical drying, can be utilized. The drying may be performed in combination with heating, for example, vacuum heating.

The step of introducing the trapping agent 3 may be performed after drying the polymer gel sheet. In such a case, after the porous sheet 2 with a plurality of cavities is formed, the trapping agent 3 is held in the cavities. For example, the porous sheet 2 is prepared by washing the polymer gel sheet with the rinse, immersing the polymer gel sheet in a predetermined solvent, such as tert-butanol or acetic acid, to replace the solvent, and drying the polymer gel sheet by a drying method, such as freeze drying or supercritical drying. The trapping agent 3 can be introduced, for example, by bringing a solution containing the trapping agent 3 into contact with the porous sheet 2. The specific mode of the contact and the step of removing unnecessary components after the contact may be the same as described above.

### [Chemical Substance Sensor]

From a different aspect, the present disclosure provides

a chemical substance sensor including a member that can trap a chemical substance,

wherein the member is a functional member according to the present disclosure.

FIG. 6 illustrates an example of a chemical substance sensor according to the present disclosure. A chemical substance sensor 11 in FIG. 6 includes the functional sheet 1 as a functional member. With the functional member, the chemical substance sensor 11 can detect a chemical substance with high sensitivity.

The chemical substance sensor 11 may be a living body sensor that detects a chemical substance secreted from a living body, such as a human body. The living body sensor may also be attached to a living body. In one example of an embodiment in which a chemical substance is detected in the state where the living body sensor is attached to a living body, the chemical substance is detected by irradiating the functional sheet 1 with at least one selected from the group consisting of visible light and ultraviolet light. More specifically, fluorescence emitted from the trapping agent 3 upon the light irradiation may be detected. In this example, the chemical substance can be detected without causing damage to the living body by light irradiation. The wavelength of the light emitted to the living body is desirably 300 nm or more. Furthermore, visible and ultraviolet irradiation is easy, and this can improve the convenience of detection.

The chemical substance sensor 11 in FIG. 6 has a monolayer structure of the functional sheet 1. The chemical substance sensor 11 can be placed to face a space, such as the interior of a room, for example. Examples of placement surfaces are described above. In such a case, it is possible to detect a chemical substance in the room atmosphere. Depending on the form of the functional sheet 1, it is also possible to detect the concentration distribution of a chemical substance in the space. The chemical substance sensor 11 can be placed near a living body, such as a human body, for example. The chemical substance sensor 11 may also be attached to a living body. In such a case, it is possible to detect a chemical substance secreted from the living body. Examples of placement surfaces are described above. The application and usage of the chemical substance sensor 11 are not limited to these examples.

The structure of the chemical substance sensor 11 is not limited to the example illustrated in FIG. 6. The chemical substance sensor 11 may have a layered structure of two or more layers including the functional sheet 1. The chemical substance sensor 11 may further include a member that supports the functional member. Examples of the supporting member are cases, holders, and supporting substrates that house the functional member. Examples of the supporting substrates include quartz glass sheets and resin films, such as PET films and cellophane films. The supporting substrates may have a larger thickness than the functional sheet 1. When the trapping agent 3 in a state of trapping a chemical substance emits fluorescence characteristic of the state upon excitation light irradiation, the supporting substrate may transmit at least one selected from the group consisting of the excitation light and fluorescence. The chemical substance sensor 11 may be provided with printing or a marker that designates a specific surface, such as a surface to be attached to a living body, a surface to be exposed to a chemical substance, or a surface to be irradiated with light for detection.

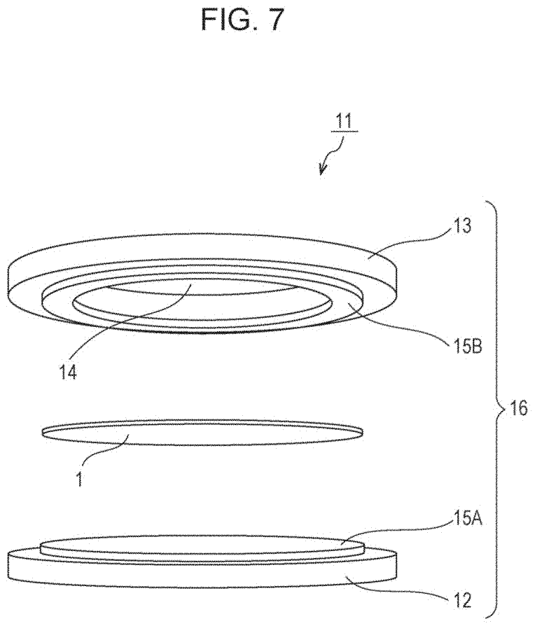

FIG. 7 illustrates one example of the chemical substance sensor 11 further including a case. FIG. 7 is an exploded perspective view of the example. The chemical substance sensor 11 in FIG. 7 further includes a case 16 that houses the functional sheet 1 as a functional member. The case 16 includes a main body 12 as a first member and a lid portion 13 as a second member. The case 16 has a through-hole 14, which is a flow path between the outside of the case 16 and the functional sheet 1 housed in the case 16. The through-hole 14 is a flow path of a fluid containing a chemical substance to be trapped. The fluid is typically a gas, such as air. The through-hole 14 is provided in the lid portion 13. In the chemical substance sensor 11 illustrated in FIG. 7, a chemical substance can be introduced into the case 16 including the functional sheet 1 through the through-hole 14. This enables the functional sheet 1 to trap and detect a chemical substance.

The main body 12 and the lid portion 13 have a magnet 15A and a magnet 15B, respectively. The magnet 15A is located on a surface of the main body 12 facing the lid portion 13. The magnet 15B is located on a surface of the lid portion 13 facing the main body 12. The magnets 15A and 15B function as a mechanism for fixing the main body 12 and the lid portion 13 to each other while the functional sheet 1 is housed between the main body 12 and the lid portion 13. In other words, the main body 12 and the lid portion 13 are fixed by the magnetic force of the magnets 15A and 15B and constitute the chemical substance sensor 11 including the functional sheet 1. The main body 12 and the lid portion 13 may be fixed by another separable means, for example, screwing or fitting, or may be fixed in an inseparable manner. Fixation with the magnets 15A and 15B, however, allows the main body 12 and the lid portion 13 to be relatively easily separated and makes it easy to replace the functional sheet 1, for example. The position where the magnets 15A and 15B are arranged is not limited to the example illustrated in FIG. 7. Furthermore, at least one member selected from the group consisting of the main body 12 and the lid portion 13 may have a mechanism for fixing the main body 12 and the lid portion 13 to each other, such as a magnet. For example, at least one member selected from the group consisting of the main body 12 and the lid portion 13 may have a mechanism for fixing these members to each other by the magnetic force of a magnet.

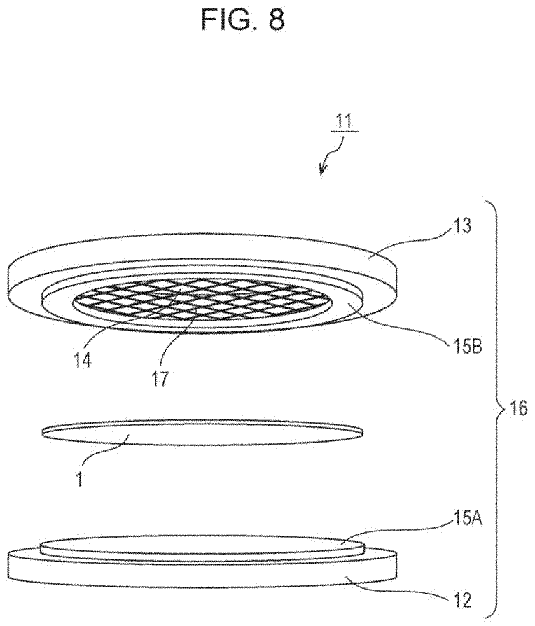

FIG. 8 illustrates another example of the chemical substance sensor 11 further including a case. FIG. 8 is an exploded perspective view of the other example. The chemical substance sensor 11 in FIG. 8 has the same structure as the chemical substance sensor 11 illustrated in FIG. 7 except that a mesh 17 is provided to cover the opening of the through-hole 14. In the chemical substance sensor 11 illustrated in FIG. 8, the mesh 17 can protect the functional sheet 1 from contact with foreign materials flying from the outside and with external objects. In other words, the chemical substance sensor 11 may further include a protective member for the functional member.

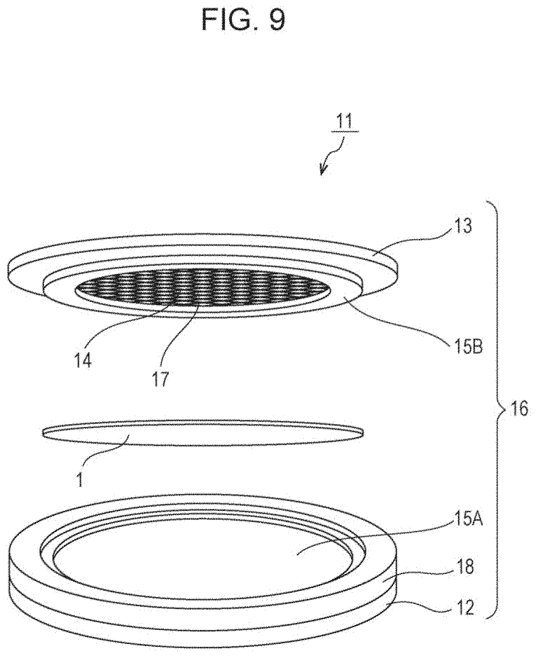

FIG. 9 illustrates another example of the chemical substance sensor 11 further including a case. FIG. 9 is an exploded perspective view of the other example. The chemical substance sensor 11 in FIG. 9 has the same structure as the chemical substance sensor 11 illustrated in FIG. 8 except that a fixing member 18 is further included. The fixing member 18 is located on a surface of the main body 12 facing the lid portion 13 and has a ring shape surrounding the magnet 15A when viewed perpendicularly to the surface. The ring typically has an inner diameter larger than the outer diameter of the magnet 15B located on the lid portion 13. When the main body 12 and the lid portion 13 are fixed with the magnets 15A and 15B, the fixing member 18 can prevent the lid portion 13 from slipping laterally and falling off. The shape and arrangement of the fixing member 18 for preventing the lid portion 13 from slipping laterally are not limited to the example illustrated in FIG. 9.

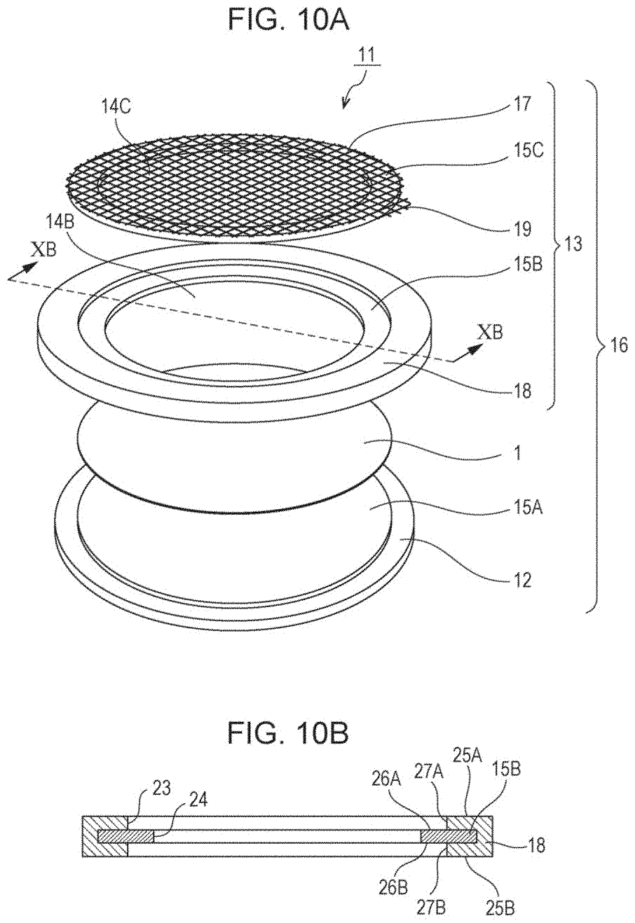

FIG. 10A illustrates another example of the chemical substance sensor 11 further including a case. FIG. 10A is an exploded perspective view of the example. FIG. 10B illustrates a cross section of a fixing member 18 and a magnet 15B in the chemical substance sensor 11 of FIG. 10A taken along the line XB-XB. The chemical substance sensor 11 in FIG. 10A further includes a case 16 that houses the functional sheet 1 as a functional member. The case 16 includes a main body 12 as a first member and a lid portion 13 as a second member. The main body 12 has a disk-shaped magnet 15A. The magnet 15A is located on a surface of the main body 12 facing the lid portion 13. The lid portion 13 includes the fixing member 18, the magnet 15B, a magnet 15C, and a mesh 17. The fixing member 18 and the magnet 15B have a ring shape. As illustrated in FIG. 10B, the magnet 15B is integrated with the fixing member 18 such that an inner circumference 24 of the magnet 15B protrudes toward the inside of the ring relative to an inner circumference 23 of the fixing member 18 and such that the magnet 15B is positioned between an upper surface 25A and a lower surface 25B of the fixing member 18. A step 27A is formed between the upper surface 25A of the fixing member 18 and an upper surface 26A of the magnet 15B, and a step 27B is formed between the lower surface 25B of the fixing member 18 and a lower surface 26B of the magnet 15B. When the main body 12 and the lid portion 13 are fixed with the magnets 15A and 15B, the functional sheet 1 can be held between the magnets 15A and 15B, and the lid portion 13 can be prevented from slipping laterally and falling off. The fixing member 18 typically has an inner diameter larger than the diameter of the magnet 15A. The magnets 15A and 15B are typically superposed with each other when viewed perpendicularly to the surface of the functional sheet 1. The step 27B may have a height equal to or smaller than the thickness of the magnet 15A to more securely hold the functional sheet 1.

The magnet 15C has a ring shape. The mesh 17 is provided to cover the opening of a through-hole 14C of the magnet 15C. The mesh 17 in FIG. 10A is positioned on the upper surface of the magnet 15C. The mesh 17 can be detachably fixed to the fixing member 18 by the magnetic force of the magnets 15B and 15C. When the mesh 17 is fixed to the fixing member 18, a fluid containing a chemical substance can flow through the mesh 17, the through-hole 14C, and the through-hole 14B of the fixing member 18. When a chemical substance is trapped, the mesh 17 can be fixed to protect the functional sheet 1 from external foreign materials or the like. On the other hand, when a chemical substance trapped by the functional sheet 1 is detected, the mesh 17 can be removed to improve detection efficiency. The mesh 17 that can be detachably fixed is particularly suitable for the detection of a chemical substance based on excitation light irradiation and fluorescence generated by the irradiation. Furthermore, the mesh 17 that can be attached and detached without separating the main body 12 from the fixing member 18 can also contribute to an improvement in detection efficiency.

The magnet 15C can be fixed to the upper surface 26A of the magnet 15B by utilizing the step 27A. Considering this, the magnet 15C may have an outer diameter smaller than the inner diameter of the magnet 15B. The mesh 17 in FIG. 10A has a tab 19 that protrudes outward from the outer circumference of the magnet 15C when viewed perpendicularly to the upper surface of the magnet 15C. The embodiment including the tab 19 is suitable for easy attachment and detachment of the mesh 17.

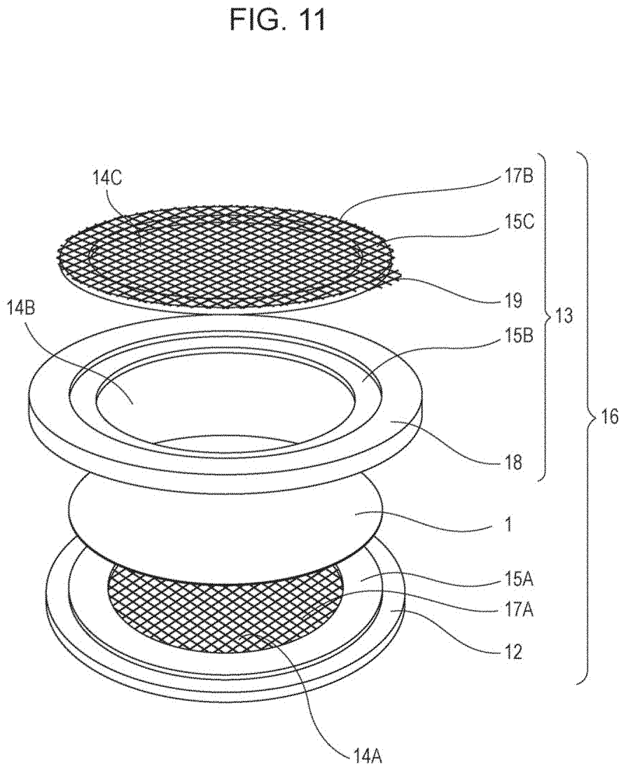

FIG. 11 illustrates another example of the chemical substance sensor 11 further including a case. FIG. 11 is an exploded perspective view of the example. The chemical substance sensor 11 in FIG. 11 has the same structure as the chemical substance sensor 11 illustrated in FIG. 10A except that the main body 12 and the magnet 15A have a through-hole 14A and a mesh 17A is provided to cover the flow cross section of the through-hole 14A. The mesh 17 in FIG. 10A is referred to as a mesh 17B in FIG. 11. For example, when the chemical substance sensor 11 is placed such that the through-hole 14C faces a living body, such as a human body, the embodiment illustrated in FIG. 11 is suitable for evaporating water vapor contained in gas emitted from the living body through the through-hole 14A and preventing condensation due to the evaporation. The mesh 17A in FIG. 11 is located between the main body 12 and the magnet 15A. The arrangement method of the mesh 17A is not limited to this example.

The chemical substance sensor 11 further including a case is not limited to these examples. For example, the flow path of a fluid containing a chemical substance may be provided in the main body 12 or in both the main body 12 and the lid portion 13. The shape of the through-hole 14 and the number of through-holes 14 are not limited to these examples. A protective member in the opening of the through-hole 14 is not limited to the mesh 17 and may be nonwoven fabric, wire mesh, net, or perforated metal, for example.

The chemical substance sensor 11 may include any member other than those described above. For example, a cover that closes the opening of the through-hole 14 may be further provided.

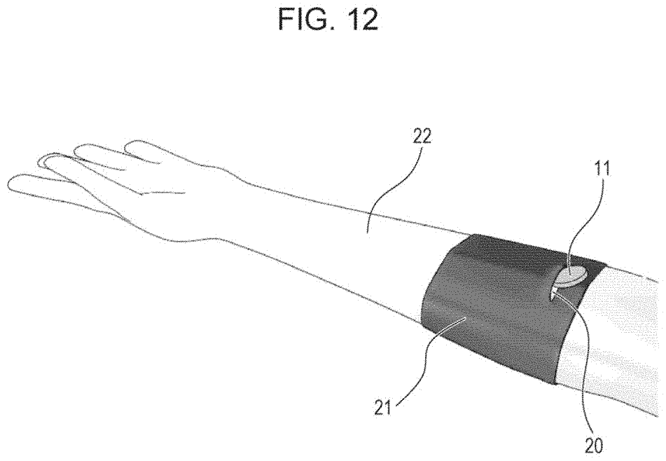

For example, the chemical substance sensors 11 illustrated in FIGS. 7 to 11 can be worn on a human body with a band or an adhesive tape or can be fixed to an object. An example of wearing on a human body is illustrated in FIG. 12. FIG. 12 is a schematic view of the example. In the example illustrated in FIG. 12, the chemical substance sensor 11 is placed in a pocket 20 of a band 21 wound around a human forearm 22. The band 21 may have gas permeability. In such a case, the chemical substance sensor 11 can more reliably detect a chemical substance secreted from a human body. The band 21 may have elasticity, which improves the adhesiveness of the chemical substance sensor 11 to a human body. For example, the chemical substance sensor 11 can be placed in the band 21 such that the through-hole 14 faces a human body. The pocket 20 in FIG. 12 is a slit in the band 21. The chemical substance sensor 11 can be placed in the band 21 through the slit such that the through-hole 14 faces a human body without the band 21 interposed therebetween. Furthermore, the fixing member 18 in the chemical substance sensor 11, for example, illustrated in FIG. 9, 10A, or 11 can prevent the lid portion 13 from slipping laterally while wearing. The usage of the chemical substance sensor 11 is not limited to this example.

### EXAMPLES

A functional member according to the present disclosure is described in more detail in the following examples. A functional member according to the present disclosure is not limited to these examples.

### Example 1

### [Synthesis of Organic Salt]

The following terephthalic acid bis(n-octylamine) salt was synthesized as a trapping agent. First, 1.00 g (6.02 mmol) of terephthalic acid and methanol were mixed to prepare 100 mL of a liquid mixture of terephthalic acid and methanol. Next, 1.95 g (15.05 mmol) of n-octylamine was poured into the liquid mixture at room temperature. The liquid mixture was then stirred at room temperature, and methanol was then evaporated under reduced pressure. Diethyl ether was then added to the resulting residue and was stirred at room temperature. After vacuum filtration and drying, 2.49 g (5.86 mmol) of a powdered terephthalic acid bis(n-octylamine) salt was produced.

### [Preparation of Methanol Solution of Organic Salt]

2.49 g of the terephthalic acid bis(n-octylamine) salt thus prepared was transferred to a volumetric flask with an internal volume of 50 mL and was diluted with methanol to prepare a methanol solution with a concentration of 5% by weight.

### [Preparation of Functional Sheet]

A regenerated cellulose membrane (RC55 manufactured by Whatman, pore size: 0.45 μm) was prepared as a porous sheet. The pore size of the porous sheet is a catalog value. The porous sheet was then placed in a beaker with an internal volume of 100 mL. The prepared methanol solution of the organic salt was poured into the beaker to immerse the porous sheet in the solution. After the immersion for 1 minute, the porous sheet was taken out, was placed on a round pinholder (manufactured by Ishizaki Kenzan, with BP Nakamaru rubber, 71 mm in diameter), and was dried under reduced pressure for 1 hour to prepare a functional sheet. The functional sheet had a disk shape with a diameter of 47 mm and a thickness of 75 μm. The weight of the functional sheet was higher by 9.8 mg than the weight of the prepared porous sheet.

### [X-Ray Diffractometry]

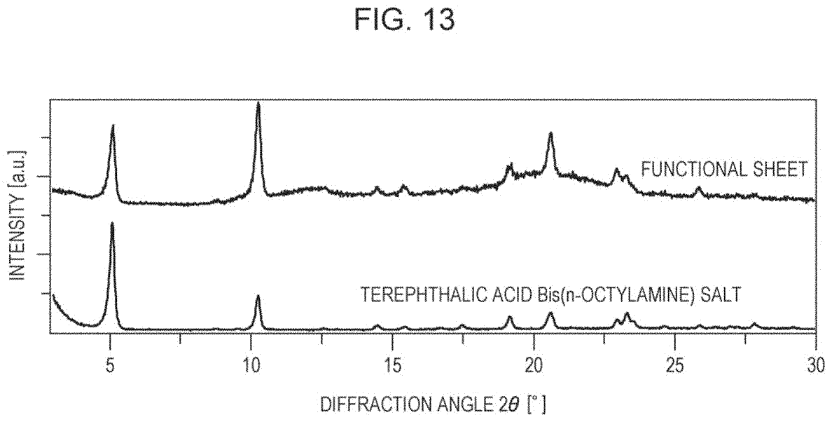

FIG. 13 shows the XRD patterns of the terephthalic acid bis(n-octylamine) salt and the functional sheet thus prepared. An automated multipurpose X-ray diffractometer (Rigaku Corporation, Ultima IV) was used for the XRD. The XRD was performed by a reflection method. The same apparatus and method were used for the XRD in the following examples and comparative examples. As shown in FIG. 13, the XRD pattern of the functional sheet had peaks at the same diffraction angles as the XRD pattern of the terephthalic acid bis(n-octylamine) salt. This means that crystal grains of the terephthalic acid bis(n-octylamine) salt are present in the functional sheet.

### [Electron Microscopic Observation]

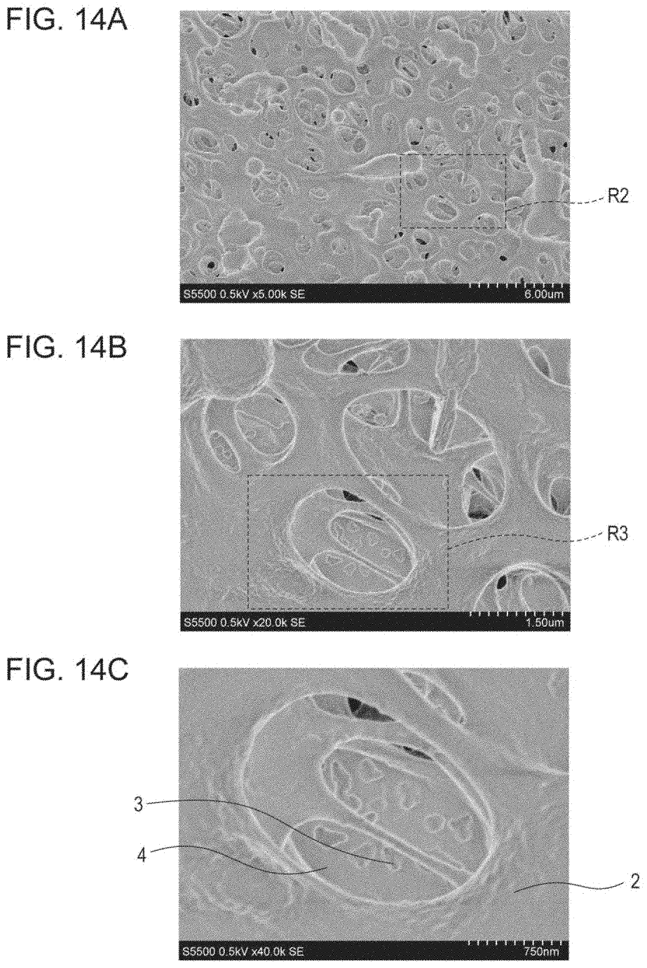

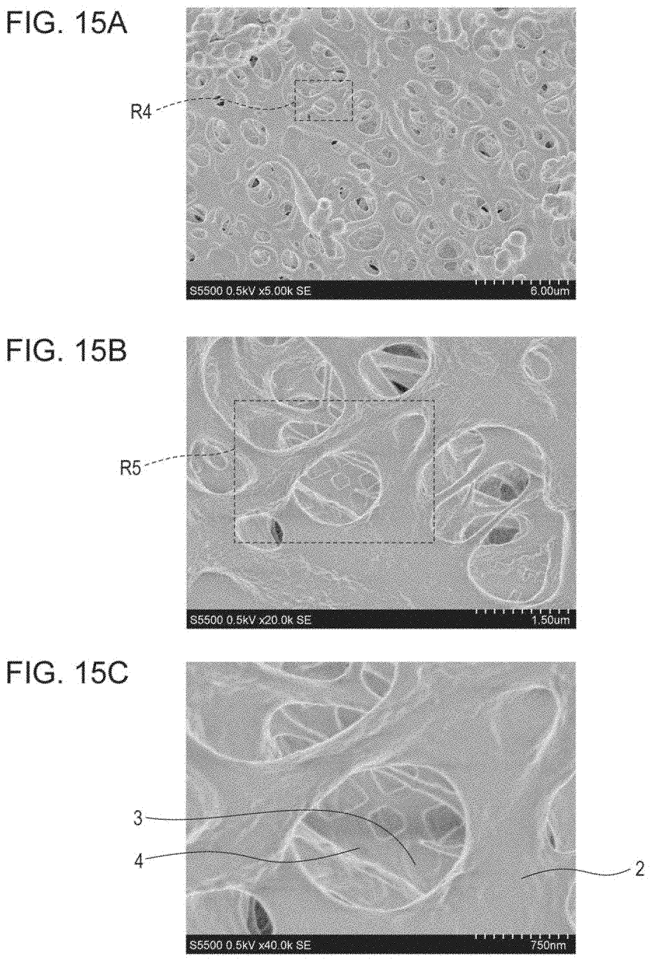

FIG. 14A shows an enlarged observation image of the prepared functional sheet acquired with a SEM (S5500 manufactured by Hitachi High-Tech Corporation). FIG. 14B shows an enlarged image of a region R2 of FIG. 14A, and FIG. 14C shows an enlarged image of a region R3 of FIG. 14B. FIG. 15A shows an enlarged observation image of another portion of the prepared functional sheet acquired with the SEM. FIG. 15B shows an enlarged image of a region R4 of FIG. 15A, and FIG. 15C shows an enlarged image of a region R5 of FIG. 15B. As shown in each figure, each of a plurality of cavities 4 in the porous sheet 2 held a large number of particles with a size smaller than the pore size of the cavities 4. The average particle size of twenty particles selected and evaluated by the above method was 0.35 μm. These particles were considered to be the trapping agent 3, that is, the terephthalic acid bis(n-octylamine) salt, held in the porous sheet 2 by immersion in the methanol solution and by subsequent drying. Thus, the functional sheet thus prepared held 9.8 mg of crystal grains of the terephthalic acid bis(n-octylamine) salt in the cavities of the porous sheet.

### [Evaluation of Hydroxyl Radical Detectability of Functional Sheet]

The hydroxyl radical detectability of the prepared functional sheet was evaluated in accordance with the following procedure.

### <Acquisition of Fluorescence Image A and Fluorescence Image A′>

The functional sheet was divided along the centerline to prepare two semicircular sheets. Each sheet thus prepared was irradiated with ultraviolet light with a wavelength of 313 nm emitted from a mercury light source (REX-250 manufactured by Asahi Spectra Co., Ltd.). Fluorescence images A and A′ of each sheet were taken with a digital camera (FLOYD manufactured by Wraymer Inc.). The fluorescence image A was the same as the fluorescence image A′. The fluorescence image A and the fluorescence image A′ are fluorescence images of the functional sheet before being exposed to an atmosphere containing hydroxyl radicals.

### <Exposure to Atmosphere Containing Hydroxyl Radicals>

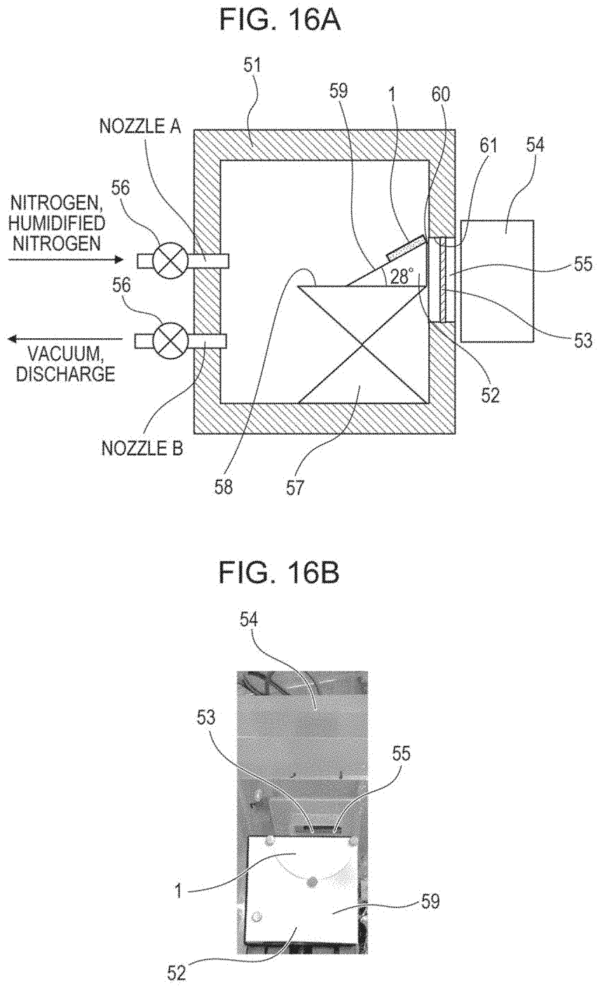

FIG. 16A illustrates a chamber used to expose a functional sheet to an atmosphere containing hydroxyl radicals. FIG. 16B shows a photograph of an actually used chamber 51 taken from a point X located obliquely above the chamber 51. The chamber 51 is made of a transparent resin, and the inside of the chamber 51 can be visually recognized from the outside. As shown in FIGS. 16A and 16B, the chamber 51 has an opening 55 on a side surface thereof. A sapphire substrate 53 is provided in the opening 55 to close the opening 55. An ozone lamp 54 that emits ultraviolet light into the chamber 51 through the opening 55 is located on a side of the opening 55 of the chamber 51. The ozone lamp 54 was GL-4Z manufactured by Kyokko Denki Co., Ltd. While the sapphire substrate 53 covering the opening 55 can seal the inside of the chamber 51, ultraviolet light with wavelengths of 254 nm and 185 nm emitted from the ozone lamp 54 can reach the inside of the chamber 51 through the sapphire substrate 53. Thus, the functional sheet 1 placed inside the sealed chamber 51 can be irradiated with ultraviolet light. The chamber 51 has a structure that can withstand a reduced pressure of one to several Torr in absolute pressure. A nozzle A and a nozzle B passing through a wall surface of the chamber 51 are provided on a side surface of the chamber 51 facing the opening 55. Nitrogen or humidified nitrogen can be charged and constantly flow into the chamber 51 through a valve 56 and the nozzle A. Furthermore, gas can be discharged from the chamber 51 through the nozzle B and a valve 56.

A jack 57 was housed in the prepared chamber 51. An inclined sample stage 52 was then placed on an upper surface 58 of the jack 57. An inclined surface 59 of the inclined sample stage 52 was inclined 28 degrees with respect to the upper surface 58 of the jack 57. The height of the jack 57 was then adjusted such that the height of a right side (the highest side of the inclined surface 59) 60 of the inclined surface 59 of the inclined sample stage 52 matched the height of an upper side 61 of the opening 55. After the fluorescence image A of the functional sheet 1 was taken, the functional sheet 1 was placed on the inclined surface 59 of the inclined sample stage 52. The semicircular sheet 1 was placed such that the chord of the semicircular sheet 1 matched the right side 60 of the inclined surface 59. Pressure reduction and subsequent nitrogen filling in the chamber 51 were repeated multiple times to replace the inside of the chamber 51 with nitrogen. Replacement with nitrogen was performed to prevent the generation of active oxygen species other than hydroxyl radicals. The amount of humidified nitrogen to be filled into the chamber 51 was then controlled such that the relative humidity in the chamber 51 ranged from 90% to 95%. The temperature in the chamber 51 was maintained in the range of 18° C. to 23° C.

After the temperature and relative humidity in the chamber 51 were stabilized, the ozone lamp 54 was turned on to irradiate the inside of the chamber 51 with ultraviolet light for 2 hours. As shown in the following formula, vacuum ultraviolet (VUV) with a wavelength of 185 nm emitted from the ozone lamp 54 cleaves a OH bond of water and generates a hydroxyl radical. The following formula is described, for example, on page 83 of “OH rajikaru rui no seisei to ouyou gijutsu (Generation and applied technology of OH radicals)” published by NTS Inc. The functional sheet was exposed to an atmosphere containing hydroxyl radicals as described above.

H₂O+VUV (185 nm)→HO.+H

### <Acquisition of Fluorescence Image B>

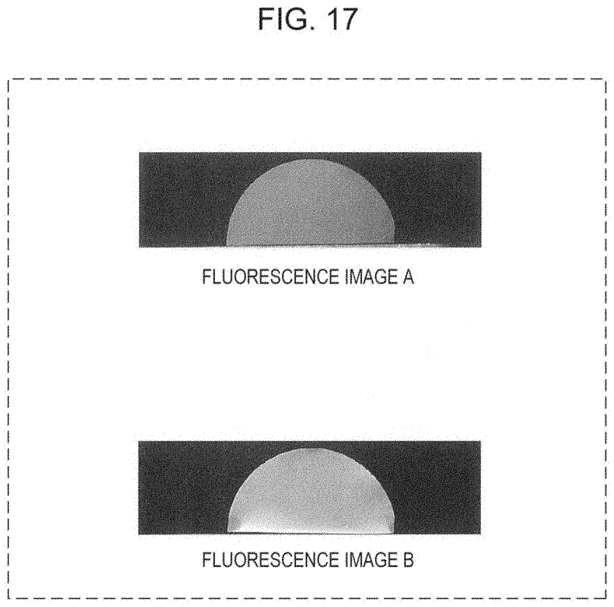

A fluorescence image B of a sheet exposed to an atmosphere containing hydroxyl radicals was taken in the same manner as the fluorescence image A. FIG. 17 shows the fluorescence image A and the fluorescence image B. As shown in FIG. 17, the functional sheet after the exposure had a higher fluorescence intensity than that before the exposure. In other words, the prepared functional sheet had hydroxyl radical detectability by an optical method. Furthermore, a particularly strong fluorescence distribution was observed at and near the chord of the semicircular sheet. The chord and the vicinity thereof were located near the opening 55 during the exposure. Thus, it is understood that vacuum ultraviolet emitted from the ozone lamp 54 had high intensity near the opening 55, and, therefore, the concentration of hydroxyl radicals increased. In other words, the functional sheet can visualize the concentration distribution of hydroxyl radicals in the space.

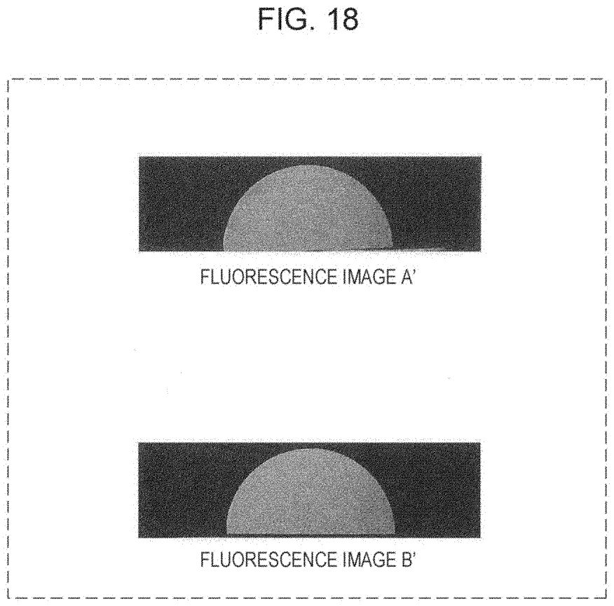

For comparison purposes, after the fluorescence image A′ of the sheet was taken, the sheet was left for 2 hours in an atmosphere maintained in the temperature range of 18° C. to 23° C. and at a relative humidity of 90% to 95% without exposure to an atmosphere containing hydroxyl radicals. A fluorescence image B′ of the sheet after being left was taken in the same manner as the fluorescence image A′. FIG. 18 shows the fluorescence image A′ and the fluorescence image B′. As shown in FIG. 18, no change of the fluorescence image B′ from the fluorescence image A′ was observed.

<Elution of Organic Salt from Functional Sheet>

A functional sheet after exposure to an atmosphere containing hydroxyl radicals was cut into fine pieces with scissors. The pieces of the sheet were then put in a screw tube bottle (No. 2 manufactured by Maruemu Corporation). Next, 2 mL of methanol was poured into the screw tube bottle to immerse the sheet in the methanol for 1 minute and elute an organic salt from the sheet, thereby preparing a methanol solution of the organic salt.

### <Fluorescence Spectrum Measurement of Methanol Solution of Organic Salt>

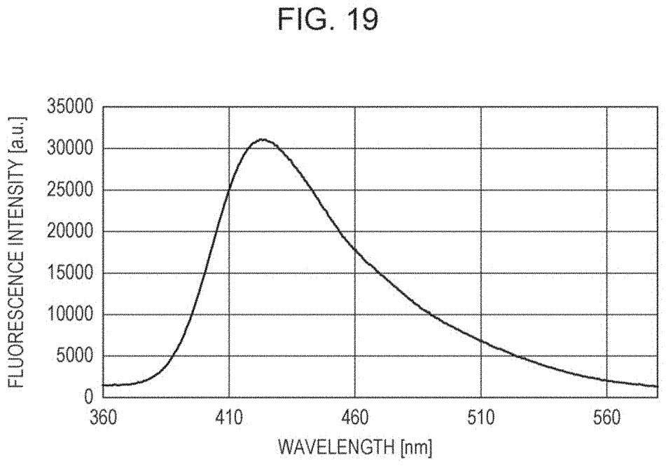

750 μL of the prepared methanol solution was transferred to a fluorescence cell (18-F/Q/10 manufactured by Pacific Science Corp.) and was subjected to fluorescence spectrum measurement. Ultraviolet light with a wavelength of 310 nm emitted from a deep ultraviolet LED (LLS 310 manufactured by Ocean Optics) was used as excitation light. Fluorescence emitted from the methanol solution upon ultraviolet irradiation was measured with a high-sensitivity spectrometer (SR-303i manufactured by Andor) as a fluorescence spectrum. FIG. 19 shows the fluorescence spectrum thus measured. As shown in FIG. 19, the emitted fluorescence had a peak at a wavelength of approximately 423 nm. This peak is not seen in a fluorescence spectrum of a terephthalic acid bis(n-octylamine) salt. On the other hand, it is described in, for example, Journal of Environmental Monitoring, 2010, 12, pp. 1658-1665 that a solution of hydroxy terephthalic acid emits fluorescence with a peak in the wavelength range of 412 to 435 nm. Thus, it was thought that part of the terephthalic acid bis(n-octylamine) salt held in the functional sheet trapped a hydroxyl radical and was converted to a hydroxy terephthalic acid bis(n-octylamine) salt. The peak intensity of the fluorescence spectrum was 31010, and this value divided by the weight 4.9 mg (=9.8/2) of the terephthalic acid bis(n-octylamine) salt held in the semicircular sheet before the exposure, that is, the peak intensity per mg of the organic salt was 6329. The peak intensity per mg of the organic salt is a measure of the hydroxyl radical detection sensitivity of the functional sheet.

### Example 2

### [Synthesis of Organic Salt]

2.49 g (5.86 mmol) of a powdered terephthalic acid bis(n-octylamine) salt was prepared in the same manner as in Example 1.

### [Preparation of Methanol Solution of Organic Salt]

A methanol solution with a concentration of 5% by weight was prepared in the same manner as in Example 1.

### [Preparation of Functional Sheet]

A functional sheet was prepared in the same manner as in Example 1 except that a hydrophilic PTFE-type membrane filter (H020A047A manufactured by Advantec Toyo Kaisha, Ltd.) was used as a porous sheet. The functional sheet had the same shape as in Example 1. The weight of the functional sheet was higher by 4.4 mg than the weight of the prepared porous sheet.

### [X-Ray Diffractometry]

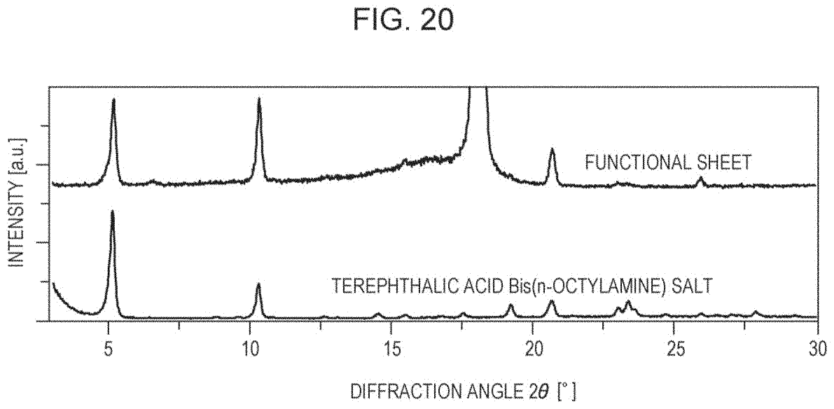

FIG. 20 shows an XRD pattern of the prepared functional sheet. As shown in FIG. 20, the XRD pattern of the functional sheet had peaks at the same diffraction angles as the XRD pattern of the terephthalic acid bis(n-octylamine) salt. This means that crystal grains of the terephthalic acid bis(n-octylamine) salt are present in the functional sheet. Thus, also in consideration of the same preparation method as the functional sheet according to Example 1, it was confirmed that the functional sheet thus prepared held 4.4 mg of crystal grains of the terephthalic acid bis(n-octylamine) salt in the cavities of the porous sheet.

### [Evaluation of Hydroxyl Radical Detectability of Functional Sheet]

The hydroxyl radical detectability of the prepared functional sheet was evaluated in accordance with the following procedure.

### <Acquisition of Fluorescence Image A and Fluorescence Image A′>

A fluorescence image A and a fluorescence image A′ of the prepared functional sheet were taken in the same manner as in Example 1. The fluorescence image A was the same as the fluorescence image A′.

### <Exposure to Atmosphere Containing Hydroxyl Radicals>

After the fluorescence image A of the sheet was taken, the sheet was exposed to an atmosphere containing hydroxyl radicals for 2 hours in the same manner as in Example 1.

### <Acquisition of Fluorescence Image B>

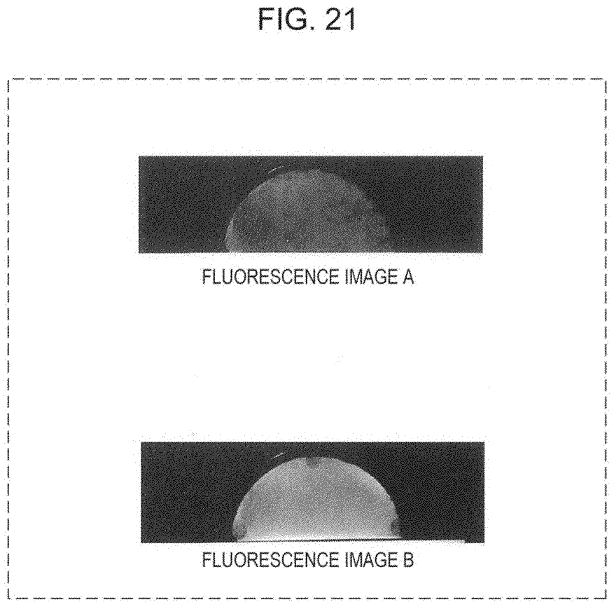

A fluorescence image B of a sheet exposed to an atmosphere containing hydroxyl radicals was taken in the same manner as the fluorescence image A. FIG. 21 shows the fluorescence image A and the fluorescence image B. As shown in FIG. 21, the functional sheet after the exposure had a higher fluorescence intensity than that before the exposure. In other words, the prepared functional sheet had hydroxyl radical detectability by an optical method. Furthermore, a particularly strong fluorescence distribution was observed at and near the chord of the semicircular sheet. The chord and the vicinity thereof were located near the opening 55 during the exposure. Thus, it is understood that vacuum ultraviolet emitted from the ozone lamp 54 had high intensity near the opening 55, and, therefore, the concentration of hydroxyl radicals increased. In other words, the functional sheet can visualize the concentration distribution of hydroxyl radicals in the space.

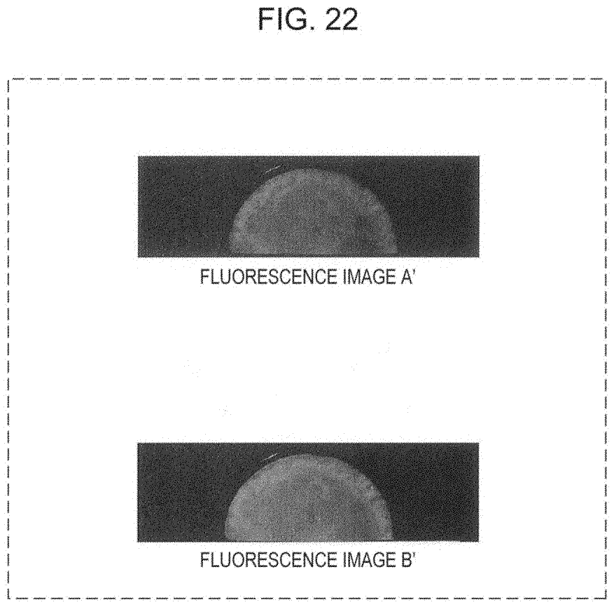

For comparison purposes, after the fluorescence image A′ of the sheet was taken, the sheet was left for 2 hours in an atmosphere maintained in the temperature range of 18° C. to 23° C. and at a relative humidity of 90% to 95% without exposure to an atmosphere containing hydroxyl radicals. A fluorescence image B′ of the sheet after being left was taken in the same manner as the fluorescence image A′. FIG. 22 shows the fluorescence image A′ and the fluorescence image B′. As shown in FIG. 22, no change of the fluorescence image B′ from the fluorescence image A′ was observed.

<Elution of Organic Salt from Functional Sheet>

A methanol solution of an organic salt was prepared in the same manner as in Example 1 from a functional sheet exposed to an atmosphere containing hydroxyl radicals and methanol.

### <Fluorescence Spectrum Measurement of Methanol Solution of Organic Salt>

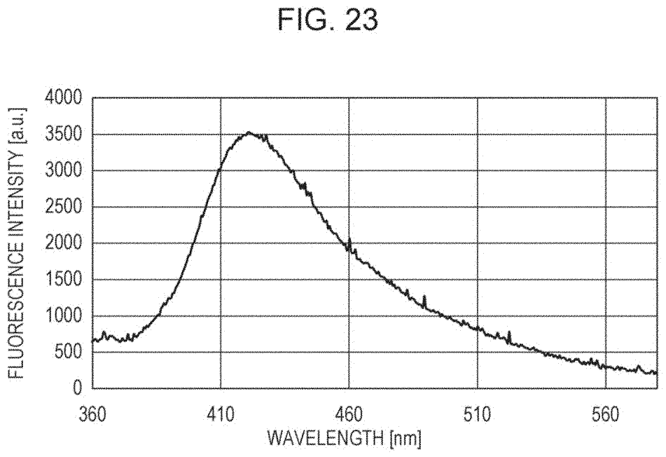

A fluorescence spectrum of the prepared methanol solution was measured in the same manner as in Example 1. FIG. 23 shows the fluorescence spectrum thus measured. As shown in FIG. 23, the emitted fluorescence had a peak at a wavelength of approximately 421 nm. Thus, it was thought that part of the terephthalic acid bis(n-octylamine) salt held in the functional sheet trapped a hydroxyl radical and was converted to a hydroxy terephthalic acid bis(n-octylamine) salt. The peak intensity of the fluorescence spectrum was 3530, and this value divided by the weight 2.2 mg (=4.4/2) of the terephthalic acid bis(n-octylamine) salt held in the semicircular sheet before the exposure, that is, the peak intensity per mg of the organic salt was 1605.

### Example 3

### [Synthesis of Organic Salt]

2.49 g (5.86 mmol) of a powdered terephthalic acid bis(n-octylamine) salt was prepared in the same manner as in Example 1.

### [Preparation of Methanol Solution of Organic Salt]

A methanol solution with a concentration of 5% by weight was prepared in the same manner as in Example 1.

### [Preparation of Functional Sheet]

A functional sheet was prepared in the same manner as in Example 1 except that a filter paper for funnel (No. 4 manufactured by Kiriyama Glass Works Co.) was used as a porous sheet. The functional sheet had the same shape as in Example 1. The weight of the functional sheet was higher by 10.4 mg than the weight of the prepared porous sheet.

### [X-Ray Diffractometry]

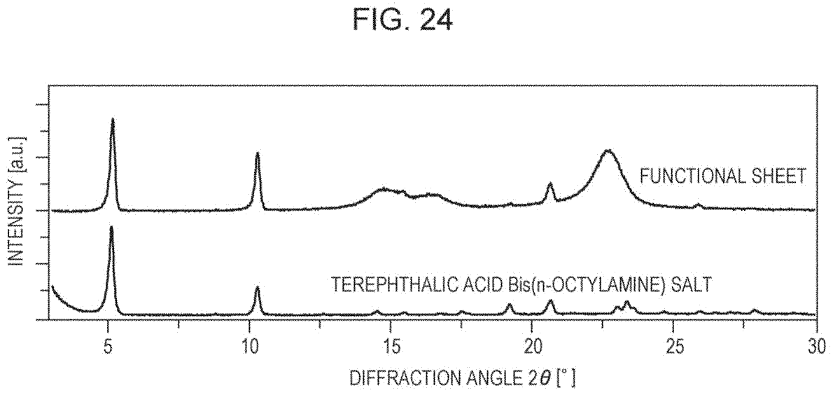

FIG. 24 shows an XRD pattern of the prepared functional sheet. As shown in FIG. 24, the XRD pattern of the functional sheet had peaks at the same diffraction angles as the XRD pattern of the terephthalic acid bis(n-octylamine) salt. This means that crystal grains of the terephthalic acid bis(n-octylamine) salt are present in the functional sheet. Thus, also in consideration of the same preparation method as the functional sheet according to Example 1, it was confirmed that the functional sheet thus prepared held 10.4 mg of crystal grains of the terephthalic acid bis(n-octylamine) salt in the cavities of the porous sheet.

### [Evaluation of Hydroxyl Radical Detectability of Functional Sheet]

The hydroxyl radical detectability of the prepared functional sheet was evaluated in accordance with the following procedure.

### <Acquisition of Fluorescence Image A and Fluorescence Image A′>

A fluorescence image A and a fluorescence image A′ of the prepared functional sheet were taken in the same manner as in Example 1. The fluorescence image A was the same as the fluorescence image A′.

### <Exposure to Atmosphere Containing Hydroxyl Radicals>

After the fluorescence image A of the sheet was taken, the sheet was exposed to an atmosphere containing hydroxyl radicals for 2 hours in the same manner as in Example 1.

### <Acquisition of Fluorescence Image B>

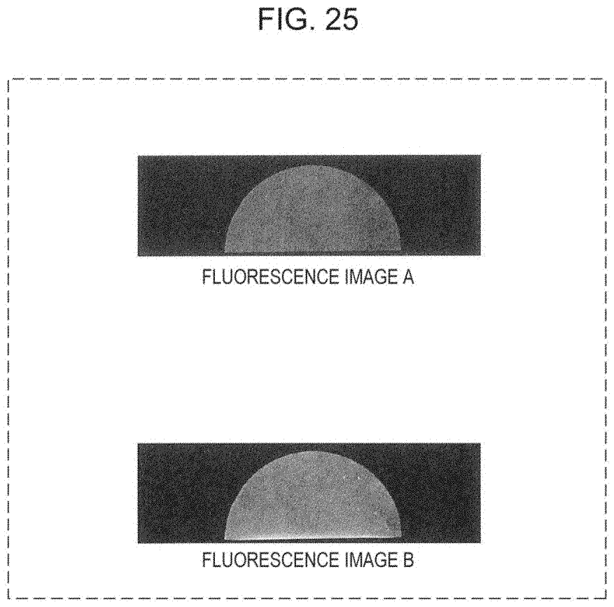

A fluorescence image B of a sheet exposed to an atmosphere containing hydroxyl radicals was taken in the same manner as the fluorescence image A. FIG. 25 shows the fluorescence image A and the fluorescence image B. As shown in FIG. 25, the functional sheet after the exposure had a higher fluorescence intensity than that before the exposure. In other words, the prepared functional sheet had hydroxyl radical detectability by an optical method. Furthermore, a particularly strong fluorescence distribution was observed at and near the chord of the semicircular sheet. The chord and the vicinity thereof were located near the opening 55 during the exposure. Thus, it is understood that vacuum ultraviolet emitted from the ozone lamp 54 had high intensity near the opening 55, and, therefore, the concentration of hydroxyl radicals increased. In other words, the functional sheet can visualize the concentration distribution of hydroxyl radicals in the space.

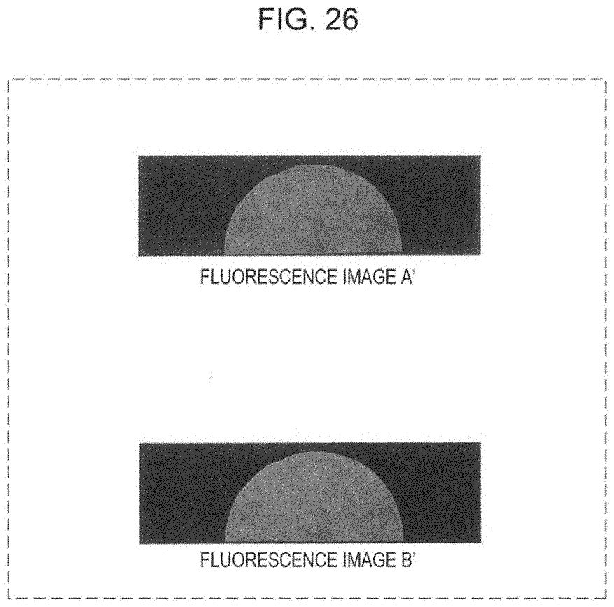

For comparison purposes, after the fluorescence image A′ of the sheet was taken, the sheet was left for 2 hours in an atmosphere maintained in the temperature range of 18° C. to 23° C. and at a relative humidity of 90% to 95% without exposure to an atmosphere containing hydroxyl radicals. A fluorescence image B′ of the sheet after being left was taken in the same manner as the fluorescence image A′. FIG. 26 shows the fluorescence image A′ and the fluorescence image B′. As shown in FIG. 26, no change of the fluorescence image B′ from the fluorescence image A′ was observed.

<Elution of Organic Salt from Functional Sheet>

A methanol solution of an organic salt was prepared in the same manner as in Example 1 from a functional sheet exposed to an atmosphere containing hydroxyl radicals and methanol.

### <Fluorescence Spectrum Measurement of Methanol Solution of Organic Salt>

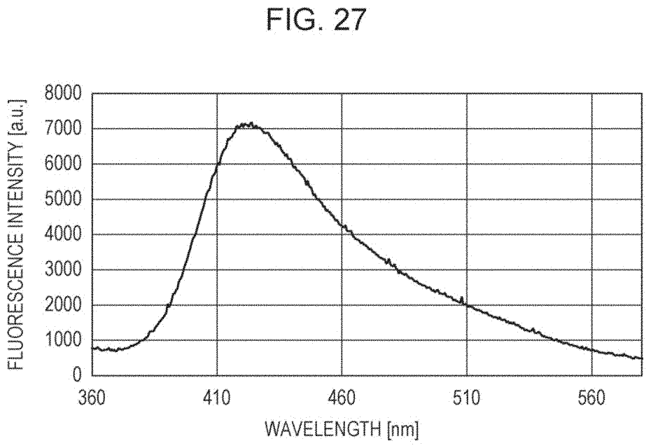

A fluorescence spectrum of the prepared methanol solution was measured in the same manner as in Example 1. FIG. 27 shows the fluorescence spectrum thus measured. As shown in FIG. 27, the emitted fluorescence had a peak at a wavelength of approximately 423 nm. Thus, it was thought that part of the terephthalic acid bis(n-octylamine) salt held in the functional sheet trapped a hydroxyl radical and was converted to a hydroxy terephthalic acid bis(n-octylamine) salt. The peak intensity of the fluorescence spectrum was 7060, and this value divided by the weight 5.2 mg (=10.4/2) of the terephthalic acid bis(n-octylamine) salt held in the semicircular sheet before the exposure, that is, the peak intensity per mg of the organic salt was 1358.

### Comparative Example 1

### [Synthesis of Organic Salt]

2.49 g (5.86 mmol) of a powdered terephthalic acid bis(n-octylamine) salt was prepared in the same manner as in Example 1.

### [Preparation of Organic Salt Pellets]

2 mg of the prepared terephthalic acid bis(n-octylamine) salt was filled in an aluminum open-type sample container (GAA-0068 manufactured by Hitachi High-Tech Science Corporation) and was pressed with a pressing machine to prepare pellets of the terephthalic acid bis(n-octylamine) salt. The pellets had a cylindrical shape with a diameter of 5.0 mm and a height of 0.5 mm.

### [Evaluation of Hydroxyl Radical Detectability of Pellets]

The hydroxyl radical detectability of the prepared pellets was evaluated in accordance with the following procedure.

### <Acquisition of Fluorescence Image A>

A fluorescence image A of a pellet thus prepared was taken in the same manner as in Example 1. However, the pellet was not divided into two.

### <Exposure to Atmosphere Containing Hydroxyl Radicals>

After the fluorescence image A of the pellet was taken, the pellet was exposed to an atmosphere containing hydroxyl radicals for 2 hours in the same manner as in Example 1. The pellet was not divided into two and was placed on the inclined surface 59 of the inclined sample stage 52. The height of the jack 57 was adjusted such that the height of the highest point of the pellet on the inclined surface 59 matched the height of the upper side 61 of the opening 55.

### <Acquisition of Fluorescence Image B>

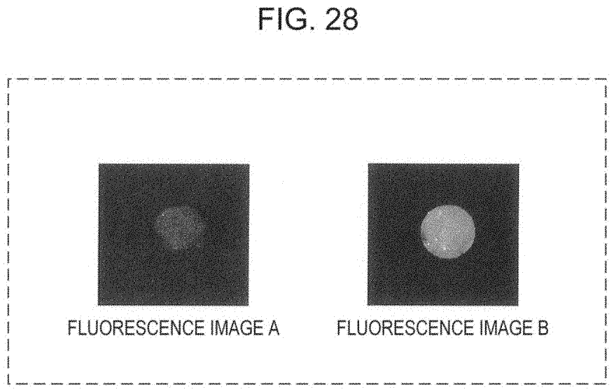

A fluorescence image B of a pellet exposed to an atmosphere containing hydroxyl radicals was taken in the same manner as the fluorescence image A. FIG. 28 shows the fluorescence image A and the fluorescence image B. As shown in FIG. 28, the pellet after the exposure had a higher fluorescence intensity than that before the exposure. In other words, the prepared pellet had hydroxyl radical detectability by an optical method.

For comparison purposes, another pellet prepared by the same method was left for 2 hours in an atmosphere maintained in the temperature range of 18° C. to 23° C. and at a relative humidity of 90% to 95% without exposure to an atmosphere containing hydroxyl radicals. A fluorescence image B′ of the pellet after being left was taken in the same manner as the fluorescence image A. The fluorescence image A and the fluorescence image B′ had no difference.

### <Dissolution of Organic Salt Pellets>

The pellet after exposure to an atmosphere containing hydroxyl radicals was put in a screw tube bottle (5-098-04 manufactured by As One Corporation). Next, 2 mL of methanol was poured into the screw tube bottle to dissolve the pellet and prepare a methanol solution of the organic salt.

### <Fluorescence Spectrum Measurement of Methanol Solution of Organic Salt>

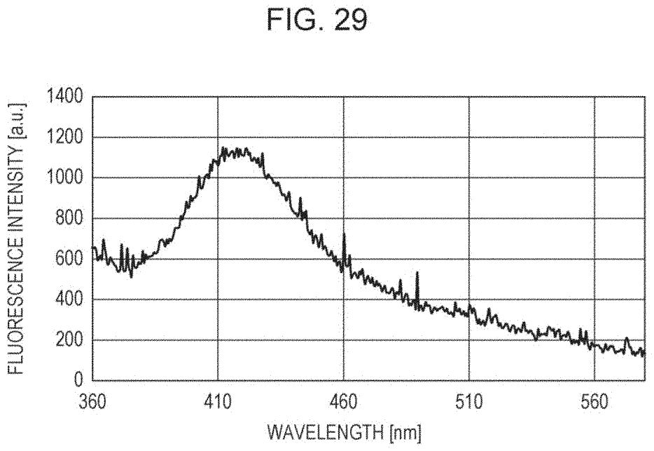

A fluorescence spectrum of the prepared methanol solution was measured in the same manner as in Example 1. FIG. 29 shows the fluorescence spectrum thus measured. As shown in FIG. 29, the emitted fluorescence had a peak at a wavelength of approximately 418 nm. Thus, it was thought that part of the terephthalic acid bis(n-octylamine) salt constituting the pellet trapped a hydroxyl radical and was converted to a hydroxy terephthalic acid bis(n-octylamine) salt. The peak intensity of the fluorescence spectrum was 1145, and this value divided by the weight 2 mg of the terephthalic acid bis(n-octylamine) salt in the pellet, that is, the peak intensity per mg of the organic salt was 573.

Table 1 summarizes the fluorescence peak intensity per mg of the terephthalic acid bis(n-octylamine) salt in Examples 1 to 3 and Comparative Example 1. Table 1 shows that the functional sheets according to Examples 1 to 3 had higher hydroxyl radical detection sensitivity than the pellets according to Comparative Example 1.

TABLE 1

| | Fluorescence peak intensity |
|---|---|
| | per mg of organic salt |
| Example 1 | 6329 |
| Example 2 | 1605 |
| Example 3 | 1358 |
| Comparative example 1 | 573 |

### Example 4

### [Exposure Test 1 to Atmosphere Containing Hydroxyl Radicals]

Functional sheets 1A, 1B, 1C, and 1D were prepared in the same manner as in Example 1 except that hydrophilic PTFE-type membrane filters shown in Table 2 were used for porous sheets. Each of the porous sheets and the functional sheets had a disk shape with a diameter of 47 mm. The weight of each functional sheet was higher by 4.4, 5.0, 4.5, and 3.8 mg than the weight of each prepared porous sheet. XRD evaluation as in Example 1 showed that crystal grains of the terephthalic acid bis(n-octylamine) salt in each functional sheet were held in the cavities of the porous sheet. The pore size and porosity of each porous sheet shown in Table 2 are catalog values.

TABLE 2

| | Porous sheet | | | | Weight of |
|---|---|---|---|---|---|
| | (Hydrophilic PTFE-type membrane filter) | | | | organic |
| | Manufac- | Product | Pore size | Porosity | salt held |
| | turer | number | (μm) | (%) | (mg) |
| Functional | Advantec | H100A047A | 1.00 | 83 | 4.4 |
| sheet 1A | | | | | |
| Functional | Advantec | H050A047A | 0.50 | 79 | 5.0 |
| sheet 1B | | | | | |
| Functional | Advantec | H020A047A | 0.20 | 71 | 4.5 |
| sheet 1C | | | | | |
| Functional | Advantec | H010A047A | 0.10 | 71 | 3.8 |
| sheet 1D | | | | | |

### [Evaluation of Hydroxyl Radical Detectability of Functional Sheet]

The hydroxyl radical detectability of the prepared functional sheet was evaluated in accordance with the following procedure.

### <Acquisition of Fluorescence Image A>

A fluorescence image A of a functional sheet thus prepared was taken in the same manner as in Example 1. However, the functional sheet was not divided into two.

### <Exposure to Atmosphere Containing Hydroxyl Radicals>

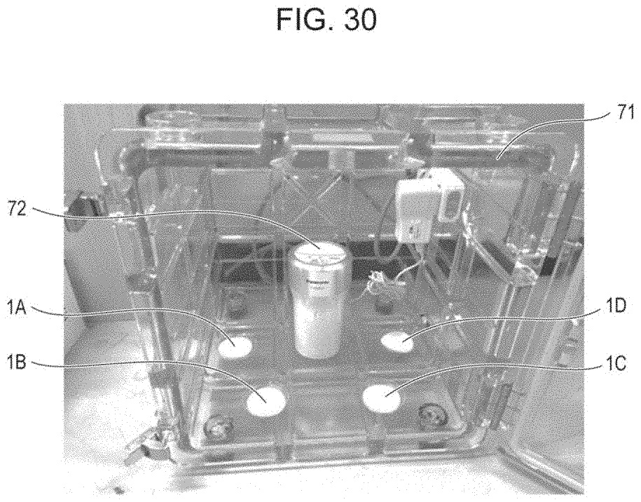

FIG. 30 illustrates a chamber used to expose a functional sheet to an atmosphere containing hydroxyl radicals and the exposure state. FIG. 30 is a photograph of an actual chamber used for the exposure and the exposure state. As shown in FIG. 30, a space cleaner 72 (nanoe generator F-GMK01 manufactured by Panasonic Corporation), which generates hydroxyl radicals, is located at the center of the floor of a chamber 71. The space cleaner 72 has the function of releasing air containing hydroxyl radicals from the upper surface thereof. The functional sheets 1A, 1B, 1C, and 1D were located at regular intervals on the floor of the chamber 71 at positions approximately 10 cm apart from the space cleaner 72. After the placement, the chamber 71 was sealed. While the inside of the chamber 71 was maintained in the temperature range of 18° C. to 23° C. and at a relative humidity of 30% to 35%, the space cleaner 72 was operated at high mode for 4 hours to expose each functional sheet to an atmosphere containing hydroxyl radicals.

### <Acquisition of Fluorescence Image B>

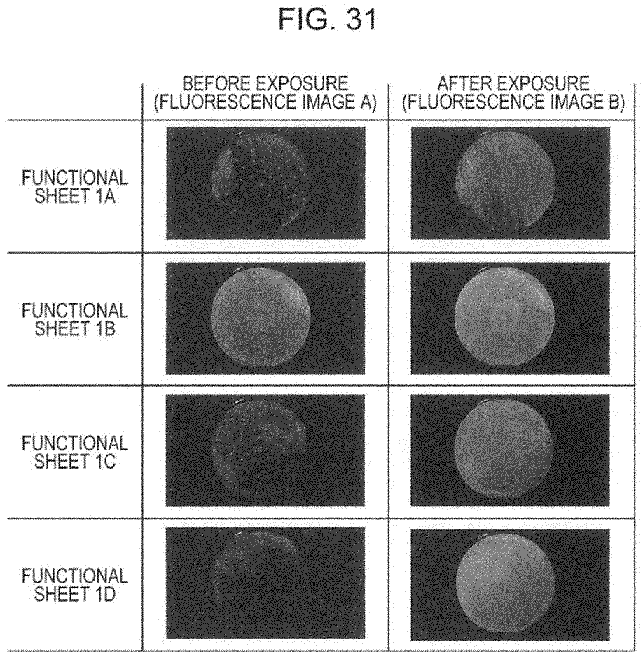

A fluorescence image B of a sheet exposed to an atmosphere containing hydroxyl radicals was taken in the same manner as the fluorescence image A. FIG. 31 shows the fluorescence image A and the fluorescence image B of each functional sheet. As shown in FIG. 31, the functional sheet after the exposure had a higher fluorescence intensity than that before the exposure. In other words, the prepared functional sheet had hydroxyl radical detectability by an optical method.

For comparison purposes, each functional sheet not exposed to the atmosphere was left for 4 hours in an atmosphere containing no hydroxyl radicals, containing ozone at a concentration of approximately 4 ppm on a volume basis, and maintained in the temperature range of 18° C. to 23° C. and a relative humidity of 30% to 35%. A fluorescence image B′ of the sheet after being left was taken in the same manner as the fluorescence image A. No change of the fluorescence image B′ from the fluorescence image A was observed.

<Elution of Organic Salt from Functional Sheet>

A methanol solution of an organic salt was prepared in the same manner as in Example 1 from a functional sheet exposed to an atmosphere containing hydroxyl radicals and methanol.

### <Fluorescence Spectrum Measurement of Methanol Solution of Organic Salt>

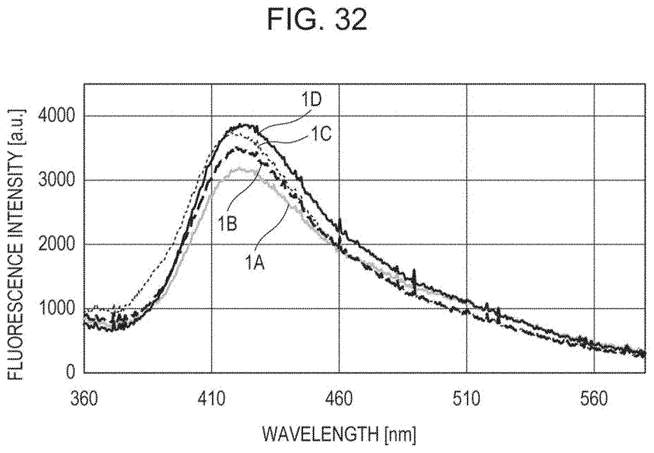

A fluorescence spectrum of the prepared methanol solution was measured in the same manner as in Example 1. FIG. 32 shows the fluorescence spectrum thus measured. As shown in FIG. 32, the emitted fluorescence had a peak at a wavelength of approximately 423 nm in the functional sheet 1A, approximately 422 nm in the functional sheet 1B, approximately 419 nm in the functional sheet 1C, and approximately 423 nm in the functional sheet 1D. It was thought that part of the terephthalic acid bis(n-octylamine) salt held in the functional sheet trapped a hydroxyl radical and was converted to a hydroxy terephthalic acid bis(n-octylamine) salt.

Table 3 summarizes the fluorescence intensity at the peak wavelength and the peak intensity per mg of the organic salt in each functional sheet. Table 3 shows that the functional sheets 1C and 1D including the porous sheets with relatively small pore sizes had higher hydroxyl radical detection sensitivity than the functional sheets 1A and 1B including the porous sheets with relatively large pore sizes.

TABLE 3

| | | | | Fluorescence spectrum | |
|---|---|---|---|---|---|
| | Porous sheet | | Weight of | Fluorescence | Peak |
| | Pore | | organic | intensity | intensity |
| | size | Porosity | salt held | at peak | per mg of |
| | (μm) | (%) | (mg) | wavelength | organic salt |
| Functional | 1.00 | 83 | 4.4 | 3167 | 723 |
| sheet 1A | | | | | |
| Functional | 0.50 | 79 | 5.0 | 3449 | 687 |
| sheet 1B | | | | | |
| Functional | 0.20 | 71 | 4.5 | 3703 | 824 |
| sheet 1C | | | | | |
| Functional | 0.10 | 71 | 3.8 | 3843 | 1017 |
| sheet 1D | | | | | |

### Example 5

### [Exposure Test 2 to Atmosphere Containing Hydroxyl Radicals]

A functional sheet 1C was prepared in the same manner as in Example 4.

### [Evaluation of Hydroxyl Radical Detectability of Functional Sheet]

The hydroxyl radical detectability of the prepared functional sheet was evaluated in accordance with the following procedure.

### <Acquisition of Fluorescence Image A>

A fluorescence image A of a functional sheet thus prepared was taken in the same manner as in Example 1. However, the functional sheet was not divided into two.

### <Exposure to Atmosphere Containing Hydroxyl Radicals>

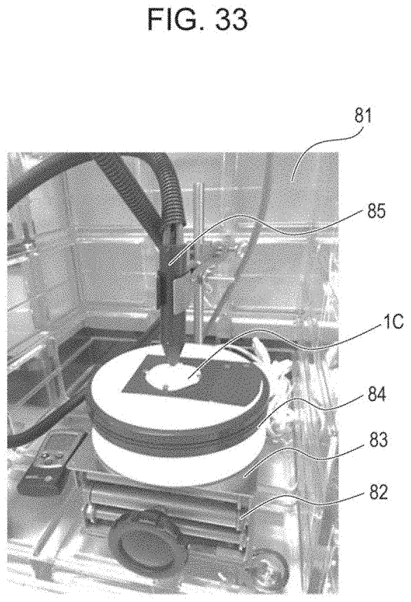

FIG. 33 illustrates a chamber used to expose a functional sheet to an atmosphere containing hydroxyl radicals and the exposure state. FIG. 33 is a photograph of an actual chamber used to expose a functional sheet to the atmosphere and the exposure state. As shown in FIG. 33, a jack 82 and a sample stage 84 on an upper surface 83 of the jack 82 are located at the center of the floor of a chamber 81. A pen-type atmospheric-pressure plasma generator (P500-SM manufactured by SAKIGAKE-Semiconductor Co., Ltd.) 85 that irradiates a functional sheet located on the sample stage 84 with plasma containing hydroxyl radicals is located above the sample stage 84. The functional sheet 1C thus prepared was placed on the sample stage 84, and the jack 82 was adjusted such that the distance between the tip of the generator 85 and the functional sheet 1C was 10 mm. The functional sheet 1C was placed on the sample stage 84 such that the tip of the generator 85 was positioned at the center of the main surface of the functional sheet 1C when viewed perpendicularly to the main surface of the sheet. After the placement, the generator 85 was operated to irradiate the functional sheet 1C with plasma containing hydroxyl radicals for 2 minutes.

### <Acquisition of Fluorescence Image B>

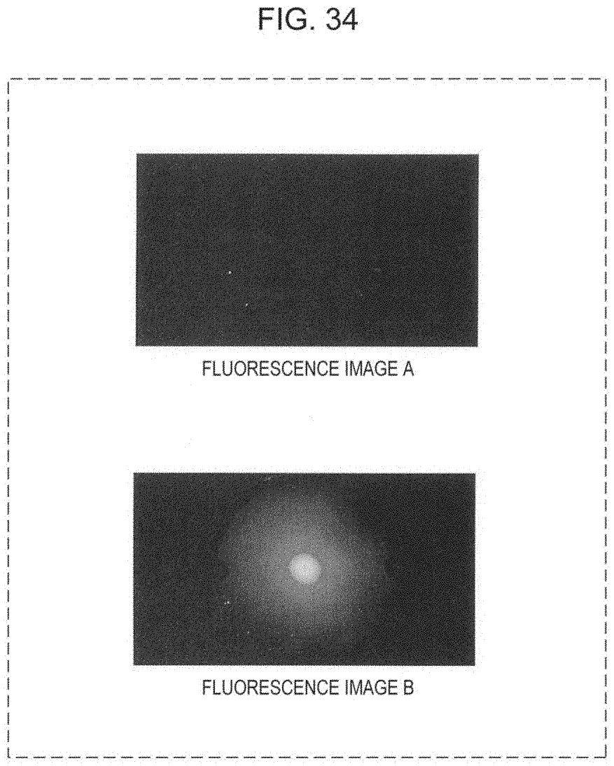

A fluorescence image B of a sheet irradiated with plasma containing hydroxyl radicals was taken in the same manner as the fluorescence image A. FIG. 34 shows the fluorescence image A and the fluorescence image B. As shown in FIG. 34, the functional sheet 1C after the exposure had a higher fluorescence intensity than that before the exposure. In other words, the prepared functional sheet 1C had hydroxyl radical detectability by an optical method. Furthermore, a particularly strong fluorescence distribution was observed near the center of the disk-shaped functional sheet 1C. The center of the functional sheet 1C was located near the tip of the generator 85 during the irradiation. It was therefore understood that the concentration of hydroxyl radicals was high near the center of the functional sheet 1C. In other words, the functional sheet 1C can visualize the concentration distribution of hydroxyl radicals in the space.

<Elution of Organic Salt from Functional Sheet>

A methanol solution of an organic salt was prepared in the same manner as in Example 1 from a functional sheet exposed to an atmosphere containing hydroxyl radicals and methanol.

### <Fluorescence Spectrum Measurement of Methanol Solution of Organic Salt>

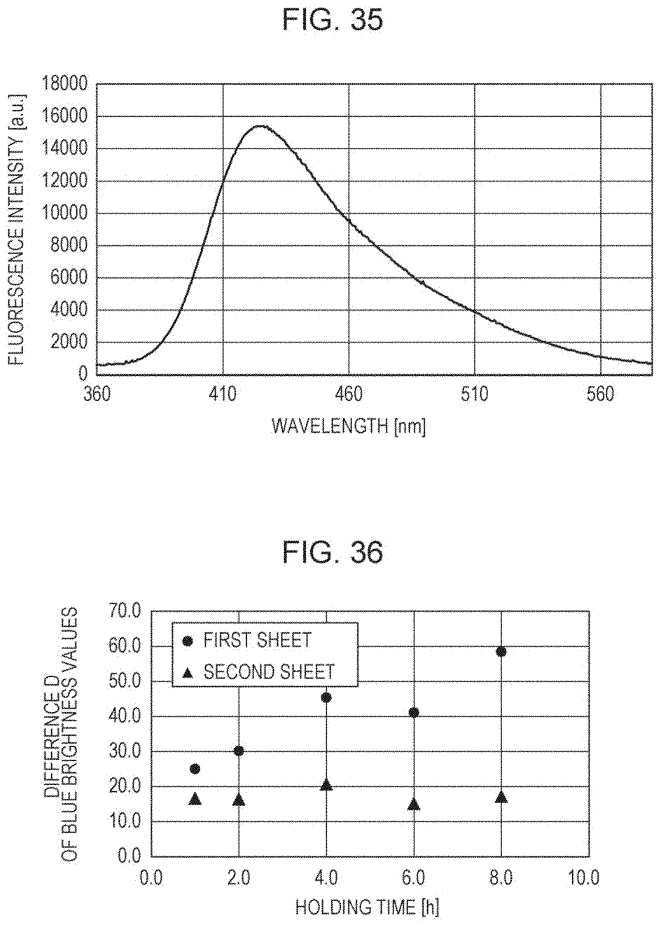

A fluorescence spectrum of the prepared methanol solution was measured in the same manner as in Example 1. FIG. 35 shows the fluorescence spectrum thus measured. As shown in FIG. 35, the emitted fluorescence had a peak at a wavelength of approximately 423 nm. It was thought that part of the terephthalic acid bis(n-octylamine) salt held in the functional sheet trapped a hydroxyl radical and was converted to a hydroxy terephthalic acid bis(n-octylamine) salt.

### Example 6

### [Body Surface Gas Exposure Test]

Five functional sheets 1C were prepared in the same manner as in Example 4. The prepared five functional sheets 1C are hereinafter referred to as sheets C1, C2, C3, C4, and C5.

The sheet C1 was divided along the centerline to prepare two semicircular sheets. One of the resulting sheets, a first sheet, was brought into contact with the surface of the palm of a subject via an ethylene-tetrafluoroethylene (ETFE) mesh that was permeable to gas in the thickness direction (AF40 manufactured by Tokyo Screen Co., Ltd.), and was left for 1 hour. The other sheet, a second sheet, was held beside the subject for 1 hour. The sheets C2 to C5 were also treated in the same manner as the sheet C1. The holding time of each sheet was 2 hours for the sheet C2, 4 hours for the sheet C3, 6 hours for the sheet C4, and 8 hours for the sheet C5.

In the same manner as in Example 1, fluorescence images of the first sheet held in contact with the subject and the second sheet held beside the subject without contact were taken before and after the holding. A difference D between the brightness values of Blue fluorescence before and after the holding was then calculated from the acquired images. The brightness value of Blue in each image was determined by image analysis as described below. Blue means B in the RGB color system. A sheet portion of each acquired image was selected by image editing software (GIMP ver. 2.8). The B values of all the pixels in the selected region were determined, and the average value of the B values was taken as the brightness value of Blue. The B value was 256 gray levels with a minimum value of 0 and a maximum value of 255.

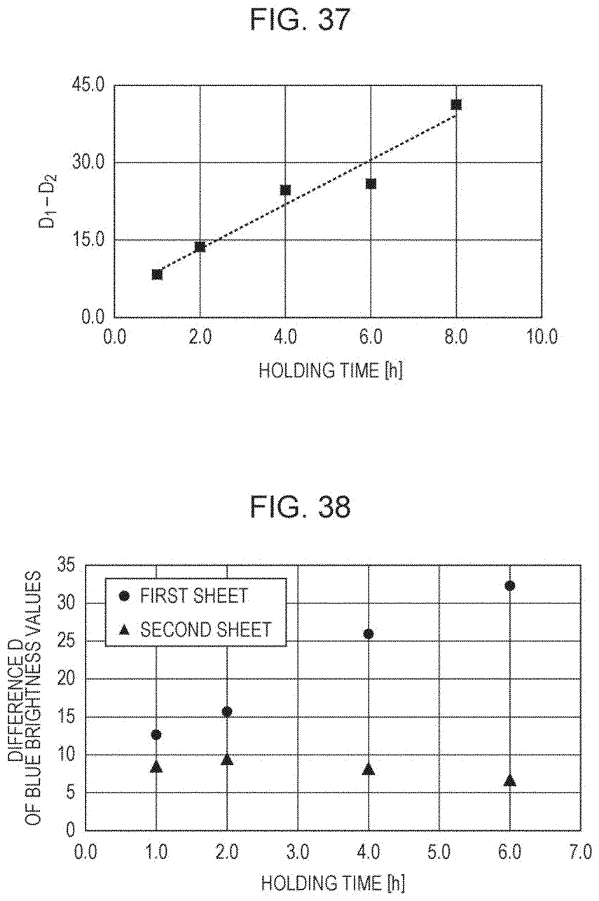

FIG. 36 is a graph in which the horizontal axis represents the holding time and the vertical axis represents the difference D between the brightness values before and after the holding. As shown in FIG. 36, in the first sheet in contact with the subject, the difference D increased with the holding time. On the other hand, in the second sheet held beside the subject, the difference D was almost constant even when the holding time increased. FIG. 37 is a graph in which the horizontal axis represents the holding time and the vertical axis represents a value D₁-D₂ obtained by subtracting the difference D₂ of the second sheet held beside the subject from the difference D₁ of the first sheet held in contact with the subject. The plot in FIG. 37 corresponds to the fluorescence properties of the functional sheet 1C, which change over time due to the effects of only a body surface gas generated from the palm. As shown in FIG. 37, the fluorescence properties changed linearly with an increase in the holding time. This showed that the body surface gas of the human body contained hydroxyl radicals, hydroxyl radicals are always released from the human body, and the fluorescence properties of the functional sheet 1C can be evaluated to quantitatively evaluate the hydroxyl radicals released from the human body. The reason for the difference D also occurred in the second sheet held beside the subject is probably due to hydroxyl radicals detected in air.

### Example 7

### [Exposure Test to Body Surface Gas]

A functional sheet 1E was prepared in the same manner as in Example 1 except that an Anopore inorganic membrane (6809-6022 manufactured by Whatman) composed of alumina was used for the porous sheet. The porous sheet and the functional sheet had a disk shape with a diameter of 25 mm and a thickness of 60 μm. The porous sheet had a pore size of 0.2 μm and a weight of 21.8 mg. The porous sheet had a porosity of 18.7% as determined from the true density of alumina of 3.95 g/cm³, the volume calculated from the diameter and thickness of the porous sheet, and the weight of the porous sheet. XRD evaluation as in Example 1 showed that crystal grains of the terephthalic acid bis(n-octylamine) salt in the functional sheet 1E were held in the cavities of the porous sheet.

The functional sheet 1E was divided along the centerline to prepare two semicircular sheets. One of the resulting sheets, a first sheet, was brought into contact with the surface of the palm of a subject via the ETFE mesh that was permeable to gas in the thickness direction (AF40 manufactured by Tokyo Screen Co., Ltd.), and was left for 2 hours. The other sheet, a second sheet, was held beside the subject for 2 hours. For both sheets, the difference D between the brightness values of Blue fluorescence before and after the holding, and the value D₁-D₂ obtained by subtracting the difference D₂ of the second sheet held beside the subject from the difference D₁ of the first sheet held in contact with the subject was calculated in the same manner as in Example 6. Table 4 shows the evaluation results for the functional sheet 1E and the evaluation results for the sheet C2 according to Example 6 with the same holding time.

TABLE 4

| | Porous sheet | | |
|---|---|---|---|
| | Pore size | Porosity | Difference |
| | (μm) | (%) | D₁ − D₂ |
| Example 7 | 0.20 | 19 | 2.1 |
| Example 6 (sheet C2) | 0.20 | 71 | 13.7 |

As shown in Table 4, in Examples 6 and 7, the difference D₁-D₂ was higher in Example 6 using the porous sheet with a larger porosity. In other words, Example 6 had a larger change in the fluorescence properties due to the effects of only the body surface gas generated from the palm. Thus, the use of the porous sheet with a larger porosity can improve the hydroxyl radical detection sensitivity of the functional sheet.

### Example 8

### [Exposure Test 2 to Body Surface Gas]

Eight functional sheets 1F were prepared in the same manner as in Example 1 except that a hydrophilic PTFE-type membrane filter (H020A025A manufactured by Advantec Toyo Kaisha, Ltd., pore size: 0.20 μm, porosity: 71%) was used for the porous sheet. The prepared eight functional sheets 1F are hereinafter referred to as the sheet F1, F1′, F2, F2′, F3, F3′, F4, and F4′. The pore size and porosity of the porous sheet are catalog values.

Next, two sets of the cases 16 illustrated in FIG. 7 were prepared. The main body 12 and the lid portion 13 of the cases 16 were composed of aluminum subjected to black alumite treatment. The main body 12 and the lid portion 13 had the magnets 15A and 15B, respectively. The main body 12 and the lid portion 13 can be fixed to each other by the magnetic force of the magnets 15A and 15B. The through-hole 14 had a circular cross-sectional shape with a diameter of 20 mm.

The sheet F1 was housed in the case 16 by placing the sheet F1 between the main body 12 and the lid portion 13 of one of the cases 16. Likewise, the sheet F1′ was housed in the other case 16. Two chemical substance sensors were fabricated in this manner.

One of the sensors, a first sensor, was then attached to a strap that mimics a wristwatch band and was worn on a forearm of a subject. The wearing was performed such that the upper surface of the lid portion 13 having the opening of the through-hole 14 came into contact with the forearm. After being left for 1 hour, the first sensor was removed from the forearm. The other sensor, a second sensor, was held beside the subject for 1 hour.

This test was also performed on a combination of the sheets F2 and F2′, a combination of the sheets F3 and F3′, and a combination of the sheets F4 and F4′. The holding time of each combination was 2, 4, and 6 hours, respectively.

In the same manner as in Example 6, for the first sensor worn by the subject and the second sensor not worn by but held beside the subject, fluorescence images of the functional sheets fixed to the respective sensors were taken before and after the holding. Excitation light irradiation and acquisition of fluorescence images were performed through the through-hole 14 of the lid portion 13. For each sensor, a difference D between the brightness values of Blue fluorescence before and after the holding was then calculated from the acquired images.

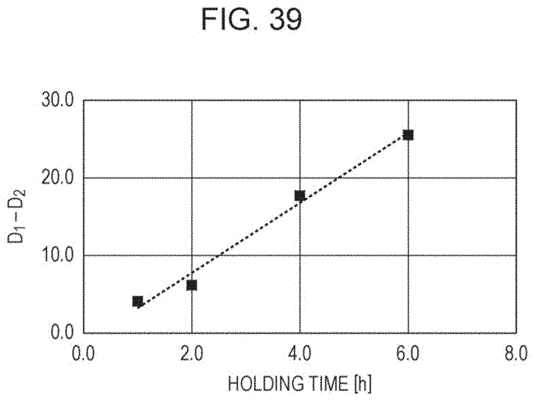

FIG. 38 is a graph in which the horizontal axis represents the holding time and the vertical axis represents the difference D between the brightness values. As shown in FIG. 38, in the first sheet in the first sensor worn by the subject and left, the difference D between the brightness values increased with the holding time. On the other hand, in the second sheet in the second sensor held beside the subject, the difference D between the brightness values was almost constant even when the holding time increased. FIG. 39 is a graph in which the horizontal axis represents the holding time and the vertical axis represents a value D₁-D₂ obtained by subtracting the difference D₂ of the second sheet in the second sensor held beside the subject from the difference D₁ of the first sheet in the first sensor held in contact with the subject. The plot in FIG. 39 corresponds to the fluorescence properties of the functional sheet 1F and the sensor including the functional sheet 1F, which change over time due to the effects of only a body surface gas generated from the palm. As shown in FIG. 39, the fluorescence properties changed linearly with an increase in the holding time. This showed that the body surface gas of the human body contained hydroxyl radicals, hydroxyl radicals are always released from the human body, and the fluorescence properties of the functional sheet 1F and the sensor can be evaluated to quantitatively evaluate the hydroxyl radicals released from the human body.

### Example 9

### [Synthesis of Organic Salt]

The following organic salt containing a cyanoacrylic acid derivative and a trisubstituted methylamine was synthesized as a trapping agent.

A three-neck flask with an internal volume of 300 mL was charged with 5.00 g (21.5 mmol) of 4-methoxy-N-phenylaniline, 5.57 g (30.1 mmol) of 4-bromobenzaldehyde, and 150 mL of toluene. Next, 0.225 g (1.00 mmol) of Pd(OAc)₂, 0.406 g (2.01 mmol) of t-Bu₃P, and 5.20 g (37.6 mmol) of potassium carbonate were added and heated with stirring and were heated under reflux for 20 hours. The product was then cooled to room temperature, and insoluble matter was removed by filtration through Celite. The filtrate was concentrated under reduced pressure. The resulting residue was then purified by silica gel column chromatography to give 5.74 g of 4-((4-methoxyphenyl)(phenyl)amino)benzaldehyde.

Next, an eggplant-shaped flask with an internal volume of 200 mL was charged with 5.73 g (18.89 mmol) of the 4-((4-methoxyphenyl)(phenyl)amino)benzaldehyde, 2.41 g (28.33 mmol) of cyanoacetic acid, and 50 mL of acetonitrile. After 3.74 mL of piperidine was poured into the flask with stirring, the reactants were heated under reflux for 1 hour. The product was then cooled to room temperature, and precipitated crystals were filtered off and suspended in 150 mL of water. Aqueous sodium carbonate was then added with stirring to adjust the pH to 10 or more, and diluted hydrochloric acid was then added to adjust the pH to 4. Crystals were filtered off and dried under reduced pressure to give 6.12 g of (E)-2-cyano-3-(4-((4-methoxyphenyl)(phenyl)amino)phenyl)acrylic acid.

The (E)-2-cyano-3-(4-((4-methoxyphenyl)(phenyl)amino)phenyl)acrylic acid and triphenylmethylamine were mixed in methanol at a mole ratio of 1:1 at room temperature. The methanol was then removed under reduced pressure to give an organic salt containing (E)-2-cyano-3-(4-((4-methoxyphenyl)(phenyl)amino)phenyl)acrylic acid and triphenylmethylamine.

### [Preparation of Mixed Solution of Organic Salt]

450 mg of the prepared organic salt was transferred to a screw tube bottle (No. 6 manufactured by Maruemu Corporation), and 7.5 mL of chloroform was added to dissolve the organic salt. Furthermore, 7.5 mL of 3-pentanone was added to prepare a mixed solution.

### [Preparation of Functional Sheet]

A filter paper for Kiriyama Rohto (funnel) (No. 4 manufactured by Kiriyama Glass Works Co.) was prepared as a porous sheet. The porous sheet was then placed in a flat laboratory dish (1-4564-03 manufactured by As One Corporation), and 5 mL of the prepared mixed solution of the organic salt was poured into the flat laboratory dish to immerse the porous sheet in the solution. After the immersion for 1 minute, the porous sheet was taken out, was placed on a round pinholder (manufactured by Ishizaki Kenzan, with BP Nakamaru rubber, 71 mm in diameter), and was dried at normal temperature and pressure for 24 hours to prepare a functional sheet. The functional sheet had a disk shape with a diameter of 21 mm and a thickness of 170 μm.

### [Preparation of Recrystallized Powder of Organic Salt]

5 mL of the mixed solution of the prepared organic salt was put into a sample tube bottle (No. 6 manufactured by Maruemu Corporation), and the sample tube bottle was allowed to stand at 35° C. for 72 hours with the lid of the sample tube bottle being half-open to prepare a recrystallized powder of the organic salt.

### [X-Ray Diffractometry]

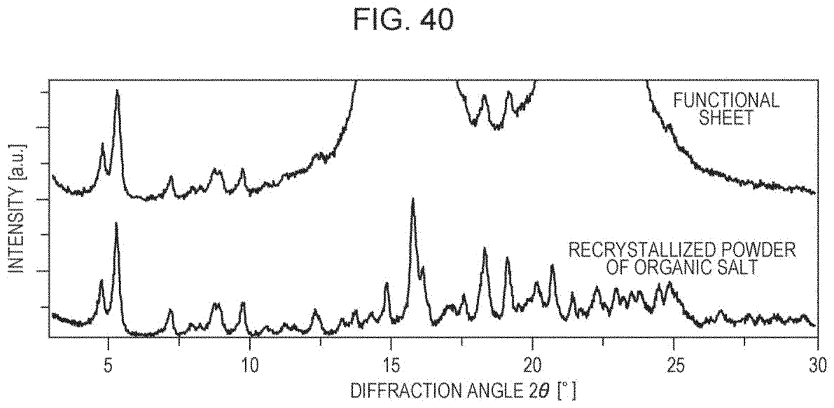

FIG. 40 shows XRD patterns of the prepared recrystallized powder and functional sheet. As shown in FIG. 40, the XRD pattern of the functional sheet had peaks at the same diffraction angles as the XRD pattern of the recrystallized powder. This means that the same crystal grains as the recrystallized powder are present in the functional sheet.

### [Evaluation of Ammonia Detectability of Functional Sheet]

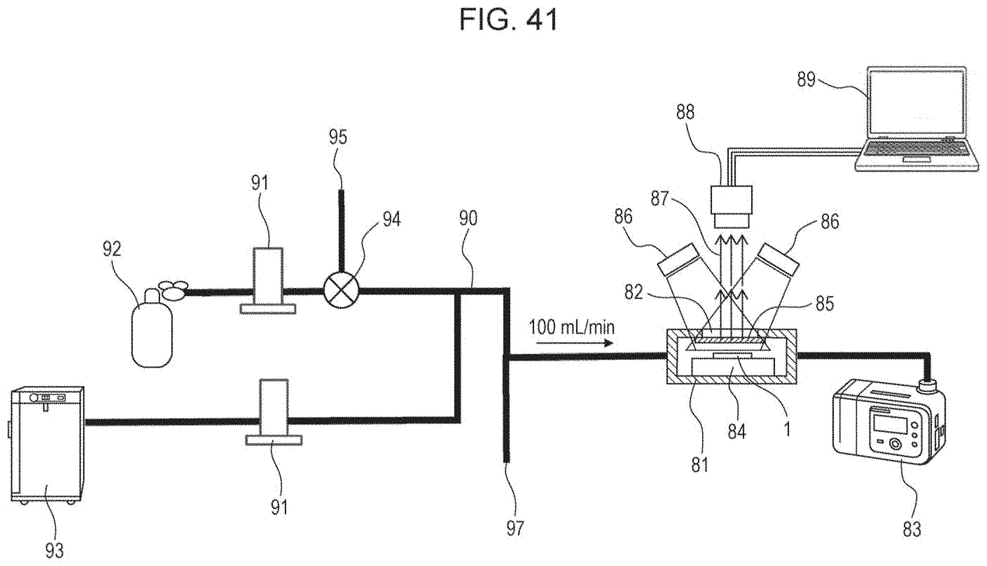

In the present example, the ammonia detectability of the prepared functional sheet was evaluated. A method of evaluating the detectability is described below with reference to FIG. 41.

A gas flow cell 81 containing the functional sheet 1 to be evaluated was prepared. The gas flow cell 81 was made of PTFE and had an opening 82 on the upper surface. The gas flow cell 81 had a through-hole on both left and right side surfaces, and dry air or ammonia gas diluted with dry air could flow into or out of the gas flow cell 81 through the through-holes. A mini-pump (MP-Σ30NII manufactured by Sibata Scientific Technology Ltd.) 83 was coupled to the through-hole on the discharge side of the gas flow cell 81. The mini-pump 83 allowed the dry air and ammonia gas diluted with dry air to flow into or out of the gas flow cell 81 at a constant flow rate.

A sample stage 84 was housed in the gas flow cell 81, and the functional sheet 1 to be evaluated was placed on the sample stage 84. A quartz substrate 85 was then placed to close the opening 82. While the inside of the gas flow cell 81 can be sealed by the quartz substrate 85, the functional sheet 1 can be irradiated with ultraviolet light with a wavelength of 365 nm emitted from an LED 86 through the quartz substrate 85. The quartz substrate 85 transmits fluorescence 87 emitted from the functional sheet 1 by the irradiation. The fluorescence can therefore be observed through the quartz substrate 85.

A digital camera (FLOYD manufactured by Wraymer Inc.) 88, with which the fluorescence could be observed, was placed directly above the opening 82. A pair of LEDs 86 for irradiating the functional sheet 1 with ultraviolet light with a wavelength of 365 nm were placed above the gas flow cell 81. The LEDs 86 were located at a position that did not interfere with the observation of fluorescence with the digital camera 88. A notebook PC 89 for processing an observed fluorescence image was coupled to the digital camera 88.

A cylinder 92 for supplying ammonia gas with a concentration of 100 ppm diluted with dry air and a compressor 93 for supplying dry air were coupled to the through-hole on the inflow side of the gas flow cell 81 through a pipe 90, a flowmeter 91, and a valve 94. A purge line 95 was coupled through the valve 94 to the pipe 90 extending from the cylinder 92. A purge line 97 for discharging excess gas that did not flow into the gas flow cell 81 was located between the gas flow cell 81 and a junction of the pipe 90 extending from the cylinder 92 and the pipe 90 extending from the compressor 93. The concentration of ammonia gas is based on volume.

Ultraviolet irradiation with the LED 86 was started, and dry air was introduced into the gas flow cell 81 with the mini-pump 83 at a flow rate of 100 mL/min for 30 minutes. During this time, ammonia gas from the cylinder 92 was discharged through the purge line 95 so as not to flow into the gas flow cell 81. The valve 94 was then operated to mix ammonia gas flowing from the cylinder 92 with dry air flowing from the compressor 93 and introduce the ammonia gas diluted to a concentration of 1000, 500, 250, or 100 ppb into the gas flow cell 81 at a flow rate of 100 mL/min for 30 minutes. The concentration of ammonia gas to be introduced was adjusted with the flowmeters 91. The valve 94 was then operated to discharge the ammonia gas flowing from the cylinder 92 through the purge line 95 and introduce only the dry air into the gas flow cell 81 at a flow rate of 100 mL/min for 30 minutes.

During the introduction of dry air or ammonia gas, fluorescence emitted from the functional sheet 1 was photographed at 30-second intervals starting from the point in time when the dry air was first introduced. The brightness value of Green was calculated from the acquired fluorescence image, and the sensor response rate was calculated using the following equation as the rate of change in the brightness of Green. The brightness value of Green in each image was determined by image analysis as described below. Green means G in the RGB color system. A sheet portion of each acquired image was selected by image editing software (GIMP ver. 2.8). The G values of all the pixels in the selected region were determined, and the average value of the G values was taken as the brightness value of Green. The G value was 256 gray levels with a minimum value of 0 and a maximum value of 255.

$$Sensor response rate = Rate of change in brightness of Green ( \% ) = \frac{Δ G}{{G}_{30}} \times 100 = \frac{{G}_{gas} - {G}_{30}}{{G}_{30}} \times 100$$

G_{gas} in the equation denotes the brightness value of Green in a fluorescence image taken during the introduction of dry air or ammonia gas. G₃₀ denotes the brightness value of Green in a fluorescence image taken at the beginning of the introduction of ammonia gas diluted to a predetermined concentration (1000, 500, 250, or 100 ppb). 30 of G₃₀ means that 30 minutes have elapsed from the point in time when the dry air was first introduced.

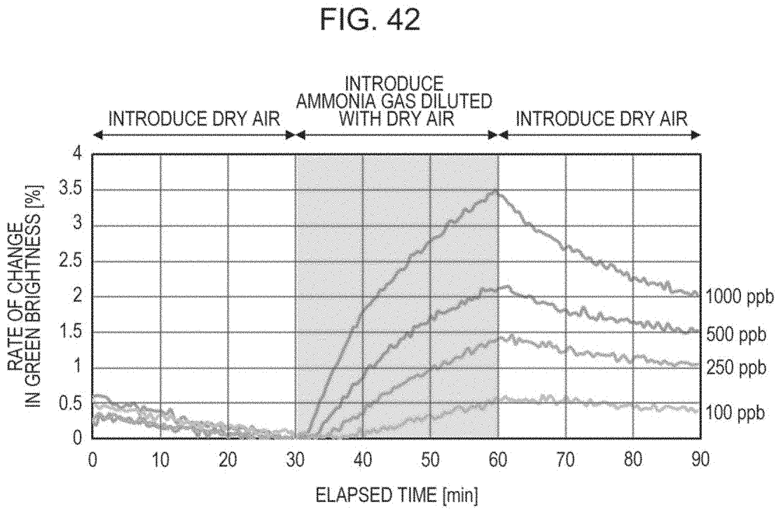

FIG. 42 shows a graph in which the horizontal axis represents the elapsed time from the point in time when the dry air was first introduced and the vertical axis represents the rate of change in the brightness of Green thus calculated. FIG. 42 shows that the functional sheet could detect ammonia gas at a very low concentration of 1000 ppb or less. It was also showed that the fluorescence properties of the functional sheet for ammonia gas changed with the concentration of the ammonia gas. In other words, the fluorescence properties of the functional sheet depend on the concentration of ammonia gas, and the functional sheet is useful as an ammonia gas sensor.

### Example 11

Cellulose derived from bleached pulp made from wood was prepared. The prepared cellulose had a purity of 80% or more. The cellulose was then sufficiently dissolved in an ionic liquid to prepare a cellulose solution. The ionic liquid contained 1-ethyl-3-methylimidazolium diethyl phosphate. The cellulose solution was applied to a surface of the substrate to form a liquid film. The application was performed by gap coating such that the target thickness of a functional sheet formed after drying was 900 nm. The substrate and the liquid film were then immersed in ethanol to remove the ionic liquid and prepare a polymer gel sheet. During the immersion, ultrasonic waves were applied at a frequency of 38 kHz and at an output of 600 W for 20 seconds or more.

Separately, 2 g (12.04 mmol) of terephthalic acid and 3.9 g (30.1 mmol) of n-octylamine were dissolved in 100 mL of ethanol to prepare an ethanol solution of a terephthalic acid bis(n-octylamine) salt as a trapping agent solution. The terephthalic acid bis(n-octylamine) salt can trap a hydroxyl radical.

The polymer gel sheet was then immersed in the trapping agent solution, and the sheet was then air-dried to prepare a functional sheet including a porous regenerated cellulose sheet and a trapping agent held in the cavities of the porous sheet. The immersion was performed for 5 minutes while shaking the solution at 10 rpm. The prepared functional sheet had a thickness of 910 nm. The thickness was determined as an average thickness at five points measured with a profiler. The profiler was Dektak manufactured by Bruker. The amount of trapping agent held in the functional sheet was 67.2% of the weight of the functional sheet.

The amount of trapping agent held in the functional sheet was determined as described below. First, the functional sheet was immersed in dimethyl sulfoxide, which was a solvent for the terephthalic acid bis(n-octylamine) salt, to extract the terephthalic acid bis(n-octylamine) salt from the functional sheet. The absorbance of the dimethyl sulfoxide solvent after the extraction was then determined with an absorptiometer at a wavelength of 250 nm. The absorptiometer was V-770 manufactured by JASCO Corporation. The absorption wavelength of 250 nm is characteristic of the terephthalic acid bis(n-octylamine) salt. The weight of the terephthalic acid bis(n-octylamine) salt in the solvent was determined from the absorbance. A calibration curve was determined in advance between the concentration of the terephthalic acid bis(n-octylamine) salt in the dimethyl sulfoxide solution of the terephthalic acid bis(n-octylamine) salt and the absorbance at 250 nm. The amount of trapping agent held in the functional sheet was then calculated from the weight of the terephthalic acid bis(n-octylamine) salt and the weight of the functional sheet.

XRD showed that the regenerated cellulose constituting the substrate of the functional sheet did not have the crystal structure I. XRD was performed on a porous sheet prepared by drying a polymer gel sheet without immersion in the trapping agent solution, that is, a porous sheet not containing the terephthalic acid bis(n-octylamine) salt. An automated multipurpose X-ray diffractometer Ultima IV manufactured by Rigaku Corporation was used for the XRD. An XRD plot obtained with CuKα radiation had no peaks at diffraction angles in the range of approximately 14 to 17 degrees and approximately 23 degrees corresponding to the crystal structure I.

The regenerated cellulose constituting the substrate of the functional sheet had a weight-average molecular weight of approximately 200,000. The weight-average molecular weight of regenerated cellulose was determined by a GPC/MALS (Multi Angle Light Scattering) method. LC-20AD manufactured by Shimadzu Corporation was used as a liquid feed unit. A differential refractometer Optilab rEX and a multi-angle light scattering detector DAWN HELEOS manufactured by Wyatt Technology Corporation were used as detectors. TSKgel α-M manufactured by Tosoh Corporation was used as a GPC column. The GPC measurement conditions include a column temperature of 23° C. and a flow rate of 0.8 mL/min. A solution prepared by dissolving a functional sheet in dimethylacetamide containing lithium chloride at a concentration of 0.1 mol/L was subjected to the GPC/MALS method.

When a portion of the prepared functional sheet was held with tweezers in the air, the functional sheet had no breakage and had the freestanding property.

The functional sheet had a visible transmittance T_{V} of 10% or more when compared with a film with a visible transmittance T_{V} of 10% serving as a limit sample. Furthermore, the visible and ultraviolet light transmittance of the functional sheet was determined with an absorptiometer. The absorptiometer was a UV visible near-infrared spectrophotometer V-770 manufactured by JASCO Corporation. The light transmittance was 43.1% for light with a wavelength of 300 nm, 56.6% for light with a wavelength of 450 nm, and 59.5% for light with a wavelength of 800 nm.

The hydroxyl radical detection sensitivity of the functional sheet was evaluated as described below. First, the functional sheet was exposed to an atmosphere containing hydroxyl radicals. The atmosphere was a nitrogen atmosphere in which an ozone lamp continuously emitted ultraviolet light with a wavelength of 185 nm. The temperature of the atmosphere was 18° C. or more and 23° C. or less, and the relative humidity was 90% or more and 95% or less. The ozone lamp was GL-4Z manufactured by Kyokko Denki Co., Ltd. The exposure time was 2 hours.

Next, the weight of the hydroxy terephthalic acid bis(n-octylamine) salt held in the functional sheet after the exposure was determined. The hydroxy terephthalic acid bis(n-octylamine) salt is formed by trapping a hydroxyl radical by the terephthalic acid bis(n-octylamine) salt, which is a trapping agent. The functional sheet after the exposure was immersed in dimethyl sulfoxide, which was a solvent for the hydroxy terephthalic acid bis(n-octylamine) salt, to extract the hydroxy terephthalic acid bis(n-octylamine) salt from the functional sheet. The dimethyl sulfoxide solvent after the extraction was then irradiated with ultraviolet light with a wavelength of 313 nm, and the intensity of fluorescence with a wavelength of 423 nm generated by the irradiation was measured. It is known that a hydroxy terephthalic acid bis(n-octylamine) salt emits fluorescence with a peak in the wavelength range of 412 to 435 nm by excitation light with a wavelength of approximately 310 nm (see S. E. Page et al., “Terephthalate as a probe for photochemically generated hydroxyl radical”, Journal of Environmental Monitoring, 2010, 12, pp. 1658-1665). REX-250 manufactured by Asahi Spectra Co., Ltd. was used as an ultraviolet light source. A spectrometer SR-303i manufactured by Andor was used to measure the amount of fluorescence. The weight of the hydroxy terephthalic acid bis(n-octylamine) salt in the solvent was then determined from the amount of fluorescence measured. A calibration curve was determined in advance between the concentration of the hydroxy terephthalic acid bis(n-octylamine) salt in the dimethyl sulfoxide solution of the hydroxy terephthalic acid bis(n-octylamine) salt and the amount of fluorescence at a wavelength of 423 nm. The ratio of the weight of the hydroxy terephthalic acid bis(n-octylamine) salt to the weight of the terephthalic acid bis(n-octylamine) salt in the functional sheet before the exposure was then determined as detection efficiency, which was a measure of the hydroxyl radical detection sensitivity of the functional sheet. The detection efficiency of the functional sheet determined by this method was 0.20%.

### Example 12

A functional sheet was prepared in the same manner as in Example 11 except that a trapping agent solution was prepared by dissolving 1 g (6.02 mmol) of terephthalic acid and 1.95 g (15.05 mmol) of n-octylamine in 100 mL of ethanol. The prepared functional sheet had a thickness of 870 nm, held the trapping agent corresponding to 52.8% of the weight of the sheet, had a light transmittance of 67.8% at a wavelength of 450 nm, and had a detection efficiency of 0.38%. The method described in Example 11 showed that the prepared functional sheet had freestanding properties and a visible transmittance T_{V} of 10% or more.

### Example 13

A functional sheet was prepared in the same manner as in Example 11 except that a trapping agent solution was prepared by dissolving 0.5 g (3.01 mmol) of terephthalic acid and 0.87 g (6.71 mmol) of n-octylamine in 100 mL of ethanol. The prepared functional sheet had a thickness of 900 nm, held the trapping agent corresponding to 52.2% of the weight of the sheet, had a light transmittance of 77.9% at a wavelength of 450 nm, and had a detection efficiency of 0.68%. The method described in Example 11 showed that the prepared functional sheet had freestanding properties and a visible transmittance T_{V} of 10% or more.

### Example 14

A functional sheet was prepared in the same manner as in Example 11 except that a trapping agent solution was prepared by dissolving 0.25 g (1.51 mmol) of terephthalic acid and 0.43 g (3.32 mmol) of n-octylamine in 100 mL of ethanol. The prepared functional sheet had a thickness of 870 nm, held the trapping agent corresponding to 31.8% of the weight of the sheet, and had a detection efficiency of 0.83%. The method described in Example 11 showed that the prepared functional sheet had freestanding properties and a visible transmittance T_{V} of 10% or more.

### Example 15

A functional sheet was prepared in the same manner as in Example 13 except that the gap thickness of the gap coating was adjusted to a target thickness of 1400 nm. The prepared functional sheet had a thickness of 1420 nm, held the trapping agent corresponding to 28.8% of the weight of the sheet, and had a detection efficiency of 1.06%. The method described in Example 11 showed that the prepared functional sheet had freestanding properties and a visible transmittance T_{V} of 10% or more.

### Example 16

A functional sheet was prepared in the same manner as in Example 13 except that an α-cellulose reagent with a purity of 95% or more was used instead of the cellulose derived from bleached pulp. In the prepared functional sheet, the regenerated cellulose constituting the porous sheet had a weight-average molecular weight of approximately 250,000, the thickness was 890 nm, the amount of the trapping agent held was 23.7% of the weight of the sheet, and the detection efficiency was 1.90%. The method described in Example 11 showed that the prepared functional sheet had freestanding properties and a visible transmittance T_{V} of 10% or more.

### Comparative Example 11

A porous sheet without the trapping agent was prepared in the same manner as in Example 11 except that the polymer gel sheet was not immersed in the trapping agent solution. The prepared porous sheet had a thickness of 920 nm and a light transmittance of 92.5% at a wavelength of 450 nm. Fluorescence emission due to ultraviolet irradiation was not observed before and after the exposure of the porous sheet to an atmosphere containing hydroxyl radicals. In other words, the porous sheet of Comparative Example 11 had a detection efficiency of 0%. The method described in Example 11 showed that the porous sheet had freestanding properties and a visible transmittance T_{V} of 10% or more.

### Comparative Example 12

After 1.00 g (6.02 mmol) of terephthalic acid was mixed with methanol, 1.95 g (15.05 mmol) of n-octylamine was further mixed, and the whole was dissolved by stirring. The methanol was then removed under reduced pressure. Diethyl ether was then added and was entirely dissolved by stirring. A powdered terephthalic acid bis(n-octylamine) salt was then prepared by vacuum filtration and drying. 1.2 mg of the powdered terephthalic acid bis(n-octylamine) salt was filled in an aluminum open-type sample container (GAA-0068 manufactured by Hitachi High-Tech Science Corporation) and was pressed with a pressing machine to prepare pellets according to Comparative Example 12. The pellets had a disk shape with a diameter of 5 mm and a thickness of 0.5 mm.

The pellets had a light transmittance of 0.1% at a wavelength of 450 nm and a detection efficiency of 0.03%.

XRD of the functional sheets according to Examples 11 to 16 and the pellets according to Comparative Example 12 showed diffraction peaks characteristic of crystals of the terephthalic acid bis(n-octylamine) salt. In other words, the functional sheets according to Examples 11 to 16 also had the crystal structure of the terephthalic acid bis(n-octylamine) salt. The automated multipurpose X-ray diffractometer Ultima IV manufactured by Rigaku Corporation was used for the XRD. The X-rays were CuKα radiation. The diffraction peaks characteristic of crystals of the terephthalic acid bis(n-octylamine) salt were observed at diffraction angles of 5, 10, and 21 degrees.

Table 5 summarizes the results according to Examples 11 to 16 and Comparative Examples 11 and 12.

TABLE 5

| | | | | | | | Comparative | |
|---|---|---|---|---|---|---|---|---|
| | Example | | | | | | example | |
| | 11 | 12 | 13 | 14 | 15 | 16 | 11 | 12 |
| Form | Sheet | | | | | | | Pellet |
| Thickness (nm) | 910 | 870 | 900 | 870 | 1400 | 890 | 920 | 500,000 |
| Trapping agent | 67.2 | 52.8 | 52.2 | 31.8 | 28.8 | 23.7 | 0 | 100 |
| content (wt %) | | | | | | | | |
| Freestanding | Yes | Yes | Yes | Yes | Yes | Yes | Yes | Yes |
| property | | | | | | | | |
| Light | 56.6 | 67.8 | 77.9 | — | — | — | 92.5 | 0.1 |
| transmittance (%) | | | | | | | | |
| (450 nm) | | | | | | | | |
| Visible | 10< | 10< | 10< | 10< | 10< | 10< | 10< | — |
| transmittance (%) | | | | | | | | |
| Detection | 0.20 | 0.38 | 0.68 | 0.83 | 1.06 | 1.90 | 0 | 0.03 |
| efficiency (%) | | | | | | | | |

Table 5 shows that the functional sheets according to Examples 11 to 16 had higher detection efficiency than Comparative Example 12. In particular, the functional sheets according to Examples 12 to 16 had 10 times or more higher detection efficiency than the pellets according to Comparative Example 12.

(Relationship between Transmittance of Light with Wavelength of 450 nm and Hydroxyl Radical Detection Efficiency)

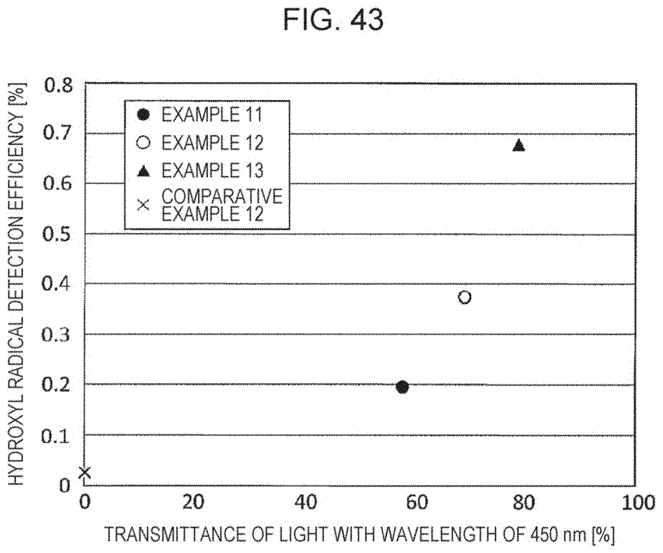

FIG. 43 shows the relationship between the transmittance of light with a wavelength of 450 nm and the hydroxyl radical detection efficiency in the functional sheets according to Examples 11 to 13 and the pellets according to Comparative Example 12. FIG. 43 shows that the hydroxyl radical detection efficiency tends to increase with the transmittance of light with a wavelength of 450 nm. Judging from the detection efficiency and this tendency, the functional sheets according to Examples 14 to 16 probably had a light transmittance of 80% or more at a wavelength of 450 nm.

### (Wearability on Human Body)

Test specimens prepared by cutting the functional sheets according to Examples 11 to 16 into a size of 2 cm×2 cm were attached to the inside skin of a human forearm using a commercial lotion. During eight hours of normal life, the functional sheets were checked for detachment from the skin and for a trouble, such as stuffiness, redness, or rash, on the attached portion of the skin. As a result, all the functional sheets were not detached from the skin and caused no skin trouble. In other words, it was confirmed that all the functional sheets could be worn for extended periods using the lotion alone and caused no stress to the skin. By contrast, the pellets according to Comparative Example 12 were immediately detached when attached to the skin using the lotion and could not be attached to the skin without a fixing means, such as an adhesive tape. Table 6 summarizes the results. Wearability to the skin was rated “good” for no detachment and “bad” for detachment during eight hours of normal life. Non-stress properties on the skin were rated “good” when no problem, such as stuffiness, redness, or rash, was observed on the attached portion during eight hours of normal life after attachment and “poor” when such a problem was observed.

TABLE 6

| | Wearability | Non-stress properties |
|---|---|---|
| | to skin | on skin |
| Example 11 | Good | Good |
| Example 12 | Good | Good |
| Example 13 | Good | Good |
| Example 14 | Good | Good |
| Example 15 | Good | Good |
| Example 16 | Good | Good |
| Comparative example 12 | Bad | Unmeasured |

### (Fluorescence Detectability on Exposed Surface and Back Surface)

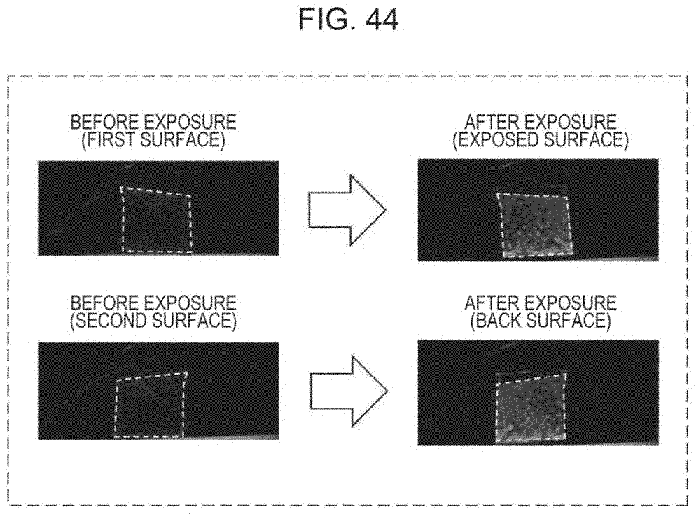

Whether fluorescence generated upon irradiation with ultraviolet light with a wavelength of 313 nm could be detected on an exposed surface of a sheet and on the back surface opposite the exposed surface was examined in the functional sheet according to Example 13 exposed to an atmosphere containing hydroxyl radicals. Whether fluorescence was observed on the test surface of the sheet placed on a quartz glass plate was examined by irradiating the test surface with ultraviolet light. The sheet to be examined was placed on the quartz glass plate such that the test surface was exposed. Both surfaces of the functional sheet according to Example 13 were also examined before exposure to the atmosphere containing hydroxyl radicals. The exposure to the atmosphere and the irradiation with ultraviolet light with a wavelength of 313 nm were performed by the methods described in Example 11. FIG. 44 shows the results. FIG. 44 shows the state of fluorescence emission due to ultraviolet irradiation in the functional sheet according to Example 13 before and after the exposure. In the sheet before the exposure to the atmosphere in FIG. 44, a surface that becomes an exposed surface after the exposure is referred to as a first surface, and a surface that becomes the back surface is referred to as a second surface.

As shown in FIG. 44, no fluorescence was observed on both surfaces of the sheet before the exposure to the atmosphere. On the other hand, fluorescence was observed on both the exposed surface and the back surface of the sheet exposed to the atmosphere. This shows that fluorescence can be observed on the back surface and that the sheet can be placed on a substrate and used as a chemical substance trapping sheet.

A functional member according to the present disclosure can be used as a chemical substance trapping member, for example. Furthermore, a sheet-like functional member according to the present disclosure can be attached to a living body, such as a human body, to detect a chemical substance secreted from the living body.

What is claimed is:

1. A functional member comprising:
a porous member with a cavity; and
a trapping agent that is held in the cavity and that traps a chemical substance.

2. The functional member according to claim 1, wherein the trapping agent has an average particle size of 1 μm or less.

3. The functional member according to claim 1, wherein the cavity has a pore size of 1 μm or less.

4. The functional member according to claim 1, wherein the porous member has a porosity of 30% or more.

5. The functional member according to claim 1, wherein the trapping agent in a state of trapping the chemical substance emits fluorescence characteristic of the state upon irradiation with excitation light.

6. The functional member according to claim 5, wherein the excitation light is ultraviolet light.

7. The functional member according to claim 1, wherein the trapping agent is an organic salt.

8. The functional member according to claim 1, wherein the chemical substance contains a hydroxyl radical.

9. The functional member according to claim 8, wherein the trapping agent is an organic salt containing terephthalic acid and at least one primary alkylamine.

10. The functional member according to claim 1, wherein the chemical substance contains ammonia.

11. The functional member according to claim 10, wherein the trapping agent is an organic salt containing a cyanoacrylic acid derivative and a trisubstituted methylamine.

12. The functional member according to claim 1, wherein
the porous member is a porous sheet with the cavity, and
the functional member is a functional sheet containing the trapping agent in the cavity of the porous sheet.

13. The functional member according to claim 12, wherein the porous sheet contains regenerated cellulose.

14. The functional member according to claim 13, wherein the regenerated cellulose has a weight-average molecular weight of 150,000 or more.

15. The functional member according to claim 12, wherein the functional sheet has a thickness of 100 nm or more and 2000 nm or less.

16. The functional member according to claim 12, wherein at least one transmittance selected from the group consisting of visible transmittance of the functional sheet and ultraviolet transmittance of the functional sheet is 10% or more and 90% or less.

17. The functional member according to claim 16, wherein the at least one transmittance is 40% or more.

18. The functional member according to claim 12, wherein the functional sheet is a biocompatible sheet.

19. A chemical substance sensor comprising the functional member according to claim 1.

20. The chemical substance sensor according to claim 19, wherein the chemical substance sensor is a living body sensor that detects the chemical substance secreted from a living body.

21. The chemical substance sensor according to claim 19, wherein the chemical substance sensor detects the chemical substance by irradiating the functional member with at least one selected from the group consisting of visible light and ultraviolet light.

22. The chemical substance sensor according to claim 19, further comprising a case that houses the functional member, wherein
the case includes a flow path that is located between an outside of the case and the functional member housed in the case, and
a fluid containing the chemical substance flows through the flow path.

23. The chemical substance sensor according to claim 22, wherein
the case includes a first member and a second member, and
at least one selected from the group consisting of the first member and the second member includes a mechanism for fixing the first member and the second member to each other while the functional member is housed between the first member and the second member.

24. The chemical substance sensor according to claim 23, wherein the mechanism fixes the first member and the second member to each other by magnetic force of a magnet.
